SEQUENCE LISTING (42 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  Description of Artificial Sequence D2E7 light chain variable region CDR3
QRYNRAPYX

SEQ ID NO: 4 - protein, 12 aa, Artificial Sequence
  Description of Artificial Sequence D2E7 heavy chain  variable region CDR3
VSYLSTASSLDX

SEQ ID NO: 5 - protein, 7 aa, Artificial Sequence
  Description of Artificial Sequence D2E7 light chain variable region CDR2
AASTLQS

SEQ ID NO: 6 - protein, 17 aa, Artificial Sequence
  Description of Artificial Sequence D2E7 heavy chain variable region CDR2
AITWNSGHIDYADSVEG

SEQ ID NO: 7 - protein, 11 aa, Artificial Sequence
  Description of Artificial Sequence D2E7 light chain variable region CDR1
RASQGIRNYLA

SEQ ID NO: 8 - protein, 5 aa, Artificial Sequence
  Description of Artificial Sequence D2E7 heavy chain variable region CDR1
DYAMH

SEQ ID NO: 9 - protein, 107 aa, Artificial Sequence
  Description of Artificial Sequence 2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  Description of Artificial Sequence 2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  Description of Artificial Sequence 2SD4 light chain variable region CDR3
QKYNSAPYA

SEQ ID NO: 12 - protein, 9 aa, Artificial Sequence
  Description of Artificial Sequence EP B12 light chain variable region CDR3
QKYNRAPYA

SEQ ID NO: 13 - protein, 9 aa, Artificial Sequence
  Description of Artificial Sequence VL10E4 light chain variable region CDR3
QKYQRAPYT

SEQ ID NO: 14 - protein, 9 aa, Artificial Sequence
  Description of Artificial Sequence VL100A9 light chain variable region CDR3
QKYSSAPYT

SEQ ID NO: 15 - protein, 9 aa, Artificial Sequence
  Description of Artificial Sequence VLL100D2 light chain variable region CDR3
QKYNSAPYT

SEQ ID NO: 16 - protein, 9 aa, Artificial Sequence
  Description of Artificial Sequence VLL0F4 light chain variable region CDR3
QKYNRAPYT

SEQ ID NO: 17 - protein, 9 aa, Artificial Sequence
  Description of Artificial Sequence LOE5 light chain variable region CDR3
QKYNSAPYY

SEQ ID NO: 18 - protein, 9 aa, Artificial Sequence
  Description of Artificial Sequence VLLOG7 light chain variable region CDR3
QKYNSAPYN

SEQ ID NO: 19 - protein, 9 aa, Artificial Sequence
  Description of Artificial Sequence VLLOG9 light chain variable region CDR3
QKYTSAPYT

SEQ ID NO: 20 - protein, 9 aa, Artificial Sequence
  Description of Artificial Sequence VLLOH1 light chain variable region CDR3
QKYNRAPYN

SEQ ID NO: 21 - protein, 9 aa, Artificial Sequence
  Description of Artificial Sequence VLLOH10 light chain variable region CDR3
QKYNSAAYS

SEQ ID NO: 22 - protein, 9 aa, Artificial Sequence
  Description of Artificial Sequence VL1B7 light chain variable region CDR3
QQYNSAPDT

SEQ ID NO: 23 - protein, 9 aa, Artificial Sequence
  Description of Artificial Sequence VL1C1 light chain variable region CDR3
QKYNSDPYT

SEQ ID NO: 24 - protein, 9 aa, Artificial Sequence
  Description of Artificial Sequence VL0.1F4 light chain variable region CDR3
QKYISAPYT

SEQ ID NO: 25 - protein, 9 aa, Artificial Sequence
  Description of Artificial Sequence VL0.1H8 light chain variable region CDR3
QKYNRPPYT

SEQ ID NO: 26 - protein, 9 aa, Artificial Sequence
  Description of Artificial Sequence LOE7.A light chain variable region CDR3
QRYNRAPYA

SEQ ID NO: 27 - protein, 12 aa, Artificial Sequence
  Description of Artificial Sequence 2SD4 heavy chain variable region CDR3
ASYLSTSSSLDN

SEQ ID NO: 28 - protein, 12 aa, Artificial Sequence
  Description of Artificial Sequence VH1B11 heavy chain variable region CDR3
ASYLSTSSSLDK

SEQ ID NO: 29 - protein, 12 aa, Artificial Sequence
  Description of Artificial Sequence VH1D8 heavy chain variable region CDR3
ASYLSTSSSLDY

SEQ ID NO: 30 - protein, 12 aa, Artificial Sequence
  Description of Artificial Sequence VH1A11 heavy chain variable region CDR3
ASYLSTSSSLDD

SEQ ID NO: 31 - protein, 12 aa, Artificial Sequence
  Description of Artificial Sequence VH1B12 heavy chain variable region CDR3
ASYLSTSFSLDY

SEQ ID NO: 32 - protein, 12 aa, Artificial Sequence
  Description of Artificial Sequence VH1E4 heavy chain variable region CDR3
ASYLSTSSSLHY

SEQ ID NO: 33 - protein, 12 aa, Artificial Sequence
  Description of Artificial Sequence VH1F6 heavy chain variable region CDR3
ASFLSTSSSLEY

SEQ ID NO: 34 - protein, 12 aa, Artificial Sequence
  Description of Artificial Sequence 3C-H2 heavy chain variable region CDR3
ASYLSTASSLEY

SEQ ID NO: 35 - protein, 12 aa, Artificial Sequence
  Description of Artificial Sequence VH1-D2.N heavy chain variable region CDR3
VSYLSTASSLDN

SEQ ID NO: 36 - DNA, 321 bp, Artificial Sequence
  Description of Artificial Sequence D2E7 light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  Description of Artificial Sequence D2E7 heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt

SEQ ID NO: 38 - protein, 800 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 39 - DNA, 2400 bp, Homo sapiens
atgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 40 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 41 - DNA, 876 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 42 - protein, 15 aa, Artificial Sequence
  Description of Artificial Sequence Synthetic peptide
GGGGSGGGGSGGGGS

(19) United States
(12) Patent Application Publication
(10) Pub. No.: US 2012/0014956 A1
(43) Pub. Date: Jan. 19, 2012

(54) METHODS AND COMPOSITIONS FOR PREDICTING RESPONSIVENESS TO TREATMENT WITH TNF-ALPHA INHIBITOR

(76) Inventors: Hartmut Kupper, Mutterstadt (DE); Hendrik Schulze-Koops, Erlangen (DE); Alla Skapenko, Munchen (DE)

(21) Appl. No.: 13/019,872

(22) Filed: Feb. 2, 2011

Related U.S. Application Data
(60) Provisional application No. 61/300,807, filed on Feb. 2, 2010.
(60) Provisional application No. 61/353,595, filed on Jun. 10, 2010.
(60) Provisional application No. 61/359,009, filed on Jun. 28, 2010.
(60) Provisional application No. 61/409,461, filed on Nov. 2, 2010.
(60) Provisional application No. 61/434,296, filed on Jan. 19, 2011.

(51) Int. Cl. A61K 39/395; A61P 29/00; A61K 38/16; C12Q 1/68; G01N 33/566

(52) U.S. Cl. 424/136.1; 435/6.11; 436/501; 424/158.1; 514/16.6; 424/133.1

(57) ABSTRACT

The invention provides methods of determining or predicting the responsiveness of a subject to treatment with a TNFα inhibitor, such as a TNFα antibody by determining genetic factors.

### REFERENCE TO RELATED APPLICATION

This application claims priority to U.S. Provisional Application No. 61/300,807, filed on Feb. 2, 2010, U.S. Provisional Application No. 61/353,595, filed on Jun. 10, 2010, U.S. Provisional Application No. 61/359,009, filed on Jun. 28, 2010, U.S. Provisional Application No. 61/409,461 filed on Nov. 2, 2010, and U.S. Provisional Application No. 61/434,296, filed on Jan. 19, 2011, the entire content of each, including the specification, any drawings, and sequence listing, are incorporated herein by reference.

### SEQUENCE LISTING

The instant application contains a Sequence Listing which has been submitted in ASCII format via EFS-Web and is hereby incorporated by reference in its entirety. Said ASCII copy, created on Feb. 1, 2011, is named 11781316.txt and is 28,619 bytes in size.

### BACKGROUND OF THE INVENTION

Rheumatoid arthritis (RA) is considered a chronic, inflammatory autoimmune disorder. RA is a disabling and painful inflammatory condition which can lead to the substantial loss of mobility due to pain and joint destruction. RA leads to the soft-tissue swelling of joints.

Conventional treatment for RA is based on methods developed for the RA patient population as a whole. As a result, known treatments may lead to some patients cycling through ineffective treatments before identifying an effective therapy. Thus, a need exists for personalized medicine to better treat RA and to identify effective treatment options for a given patient. Being able to adequately predict an RA patient's response to a therapeutic agent, would facilitate treatment. Identifying in advance patients who are likely to respond to a given therapeutic agent also allows RA patients to be treated early enough to, for example, prevent irreversible joint damage and resulting disability.

A variety of biomarkers for RA have been identified as being associated with the RA disease condition (see, for example, Poole and Dieppe (1994) Seminars in Arthritis and Rheumatism 23:17; Nakamura (2000) J Clin Lab Analysis 14:305; and Young et al. (2001) Annals Rhuematic Diseases 60:545; Rioja et al. ((2008) Arthritis & Rheum 58(8):2257). In some instances, biomarkers have also been identified as influencing the clinical efficacy of certain therapeutic antibodies. For example FCGR2A and FCGR3A polymorphisms have been found to influence the clinical efficacy of the antibody infliximab in RA patients (Canete et al. (2009) Ann Rheum Dis 68:1547; Tsukahara et al. (2008) Ann Rheum Dis 67:1791). Despite these findings, there remains a need for more effective means to determine which patients having RA will respond to various treatment options.

### SUMMARY OF THE INVENTION

The identification of a genetic marker or genetic markers that would help to predict or assess the effectiveness of a given treatment for RA remains a challenge. The present invention, at least in part, identifies three biomarkers that may be used alone, or in combination with one another, to predict whether a subject having rheumatoid arthritis will be responsive to treatment with a TNFα inhibitor. The present invention is based, at least in part, on the identification of molecular markers that can be used to assess the responsiveness of a subject to a treatment(s), e.g., prior to or concomitantly with administration of the treatment(s), e.g., human TNFa antibodies, or antigen binding portions thereof. Specifically, the present invention provides methods and compositions that can be used to determine whether a subject having an autoimmune disease, such as rheumatoid arthritis (RA) will be responsive to treatment with a TNFα inhibitor. The invention is based, at least in part, on the observation that the presence or copy number of particular alleles, e.g., HLA-DRB1 shared epitope (HLA-DRB1 SE), IL-4R I50V polymorphism, and/or FcγRIIb I232T polymorphism, in a subject is associated with increased or decreased responsiveness to treatment with a TNFα inhibitor and/or methotrexate (MTX).

Accordingly, in one aspect, the present invention provides methods for determining, predicting, or assessing responsiveness to treatment with a TNFα inhibitor in a subject having an autoimmune disorder, e.g., rheumatoid arthritis (RA), and methods for treating a subject having an autoimmune disorder, e.g., RA which include determining the genotype of the subject, wherein the genotype indicates that the subject will be responsive to treatment with the TNFα inhibitor.

In one aspect, the invention provides a method for predicting the responsiveness of a subject having an autoimmune disorder, e.g., rheumatoid arthritis (RA), to treatment with a TNFα inhibitor, the method comprising determining the presence or, e.g., the number of copies, of an HLA-DRB1 shared epitope (HLA-DRB1 SE) allele in a sample from the subject, wherein the presence of one or two copies of the HLA-DRB1 SE allele indicates that the subject will be responsive to treatment with the TNFα inhibitor.

In one aspect, the invention provides a method for treating a subject having an autoimmune disease, such as, rheumatoid arthritis (RA) comprising administering a TNFα inhibitor to the subject for the treatment of RA, provided that at least one copy, e.g., one or two copies, of an HLA-DRB1 shared epitope (HLA-DRB1 SE) allele are present in a sample from the subject.

In a related aspect, the invention provides a method for treating a subject having an autoimmune disease, such as rheumatoid arthritis (RA), the method comprising determining the number of copies of an HLA-DRB1 shared epitope (HLA-DRB1 SE) allele in a sample from the subject, and administering to the subject a therapeutically effective amount of the TNFα inhibitor, if the subject has one or two copies of the HLA-DRB1 SE allele.

In one aspect, the invention provides a method for treating a subject having an autoimmune disease, e.g., rheumatoid arthritis (RA), the method comprising determining the number of copies of an HLA-DRB1 shared epitope (HLA-DRB1 SE) allele, and the presence of an IL-4R I50 allele in a sample from the subject, and, administering to the subject a therapeutically effective amount of a TNFα inhibitor, if the subject has no HLA-DRB1 SE allele, and if the subject has at least one (preferably two) IL-4R I50 allele in the sample.

In another aspect, the present invention provides a method of determining whether a TNFα inhibitor will be effective for the treatment of a subject having an autoimmune disease, e.g., rheumatoid arthritis (RA), the method comprising detecting the presence of at least one copy of an HLA-DRB1 shared epitope (HLA-DRB1 SE) allele in a sample from the subject, wherein the presence of the HLA-DRB1 SE allele indicates that the TNFα inhibitor will be effective for the treatment of the autoimmune disease, e.g., RA, in the subject.

In one embodiment, the number of copies of the HLA-DRB1 SE allele is determined by assaying nucleic acid, e.g., DNA, or protein in the sample. In another embodiment, the number of copies of the HLA-DRB1 SE allele is determined using an assay method selected from the group consisting of microarray analysis, DNA sequencing, or PCR techniques, including, but not limited to allele-specific PCR.

In certain embodiments of the invention, the methods further comprise determining the number of copies of an IL-4R I50 allele in a sample from the subject, wherein the presence of the IL-4R I50 allele (AA or AG) in the sample indicates that the subject will be responsive to treatment with the TNFα inhibitor.

In other embodiments, the methods of the invention further comprise determining the presence of two FcγRIIb T232 alleles (FcγRIIb-CC) in a sample from the subject, wherein the presence of two FcγRIIb T232 alleles (FcγRIIb-CC) in the sample indicates that the subject will be responsive to treatment with the TNFα inhibitor.

In other embodiments, the methods of the present invention further comprise determining the number of copies of an IL-4R I50 allele in a sample from the subject and determining the presence of two FcγRIIb T232 alleles (FcγRIIb-CC) in a sample from the subject, wherein the presence of the IL-4R I50 allele (AA or AG) in the sample and the presence of two FcγRIIb T232 alleles (FcγRIIb-CC) in the sample indicates that the subject will be responsive to treatment with the TNFα inhibitor.

In one aspect, the present invention provides a method of predicting the responsiveness of a subject having an autoimmune disease, e.g., RA, to treatment with a TNFα inhibitor, the method comprising determining the copy number of an FcγRIIb T232 allele in a sample from the subject, wherein the presence of two copies of the FcγRIIb T232 allele (FcγRIIb-CC) indicates that the subject will be responsive to treatment with the TNFα inhibitor.

In another aspect, the present invention provides a method for treating a subject having an autoimmune disease, e.g., rheumatoid arthritis (RA), comprising administering a TNFα inhibitor to the subject for the treatment of the autoimmune disease, e.g., RA, provided that two copies of the FcγRIIb T232 allele (FcγRIIb-CC) are present in a sample from the subject.

In yet another aspect, the present invention provides a method of determining whether a TNFα inhibitor will be effective for the treatment of a subject having an autoimmune disease, e.g., rheumatoid arthritis (RA), the method comprising determining the copy number of an FcγRIIb T232 allele in a sample from the subject, wherein the presence of two copies of the FcγRIIb T232 allele (FcγRIIb-CC) indicates that the TNFα inhibitor will be effective for the treatment of the autoimmune disease, e.g., RA in the subject.

In one embodiment, the presence of the FcγRIIb T232 allele is determined by assaying nucleic acid, e.g., DNA, or protein in the sample. In another embodiment, the presence of the FcγRIIb T232 allele is determined using an assay method selected from the group consisting of microarray analysis, DNA sequencing, or PCR techniques, including, but not limited to allele-specific PCR.

In one aspect, the present invention provides a method of predicting the responsiveness of a subject having an autoimmune disease, e.g., rheumatoid arthritis (RA), to treatment with a TNFα inhibitor, the method comprising determining the number of copies of an IL-4R V50 allele in a sample from the subject, wherein the presence of two copies of the IL-4R V50 allele (GG) in the sample indicates that the subject will not be responsive to treatment with the TNFα inhibitor, unless the subject also has at least one copy of an HLA-DRB1 SE allele.

In one embodiment, the number of copies of the IL-4R V50 allele is determined by assaying nucleic acid, e.g., DNA, or protein in the sample. In another embodiment, the number of copies of the IL-4R V50 allele is determined using an assay method selected from the group consisting of microarray analysis, DNA sequencing, or PCR techniques, including, but not limited to allele-specific PCR.

The invention also includes a method for determining or predicting responsiveness to treatment with a TNFα inhibitor in a subject having an autoimmune disease, such as rheumatoid arthritis (RA), the method comprising determining the presence of an IL-4R I50 allele in a sample from the subject, wherein the presence of the IL-4R I50 allele (preferably two copies of the IL-4R I50 allele, e.g., a genotype of AA) in the sample indicates that the subject will be responsive to treatment with the TNFα inhibitor.

In one embodiment, the presence of the IL-4R I50 allele is determined by assaying nucleic acid, e.g., DNA, or protein in the sample. In another embodiment, the presence of the IL-4R I50 allele is determined using an assay method selected from the group consisting of microarray analysis, DNA sequencing, or PCR techniques, such as, but not limited to allele-specific PCR.

The invention also includes a method for treating a subject having an autoimmune disease, such as rheumatoid arthritis (RA), the method comprising determining the presence of an IL-4R I50 allele in a sample from the subject, and administering to the subject a therapeutically effective amount of a TNFα inhibitor, if the subject has at least one IL-4R I50 allele (e.g., genotype is AA or AG).

In one embodiment, the presence of the IL-4R I50 allele is determined by assaying nucleic acid, e.g., DNA, or protein in the sample. In another embodiment, the presence of the IL-4R I50 allele is determined using an assay method selected from the group consisting of microarray analysis, DNA sequencing, or PCR techniques, such as, but not limited to allele-specific PCR.

The present invention also provides a method of predicting the responsiveness of a subject having an autoimmune disease, e.g., rheumatoid arthritis (RA), to treatment with a TNFα inhibitor, the method comprising determining the number of copies of an HLA-DRB1 shared epitope (HLA-DRB1 SE) allele in a sample from the subject and the number of copies of an IL-4R I50 allele in a sample from the subject, wherein the presence of the HLA-DRB1 SE allele and the presence of the IL-4R I50 allele (AA or AG) in the sample indicates that the subject will be responsive to treatment with the TNFα inhibitor.

In one aspect, the present invention provides a method for treating a subject having an autoimmune disease, e.g., rheumatoid arthritis (RA), comprising administering a TNFα inhibitor to the subject for the treatment of RA, provided that one or two copies of an HLA-DRB1 shared epitope (HLA-DRB1 SE) allele and one or two copies of an IL-4R I50 allele (AA or AG) are present in a sample from the subject.

In another aspect, the present invention provides a method of determining whether a TNFα inhibitor will be effective for the treatment of a subject having an autoimmune disease, e.g., rheumatoid arthritis (RA), the method comprising detecting the presence of at least one copy of an HLA-DRB1 shared epitope (HLA-DRB1 SE) allele in a sample from the subject and the number of copies of an IL-4R I50 allele in a sample from the subject, wherein the presence of the HLA-DRB1 SE allele and the presence of one or two copies of the IL-4R I50 allele (AA or AG) indicates that the TNFα inhibitor will be effective for the treatment of RA in the subject.

In one embodiment, the presence of the IL-4R I50 allele is determined by assaying nucleic acid, e.g., DNA, or protein in the sample. In another embodiment, the presence of the IL-4R I50 allele is determined using an assay method selected from the group consisting of microarray analysis, DNA sequencing, or PCR techniques, such as, but not limited to allele-specific PCR.

In one embodiment of the invention, the subject is a human.

In another embodiment of the invention, the RA is early rheumatoid arthritis.

In still another embodiment, the method determines or predicts clinical responsiveness in the subject.

In another embodiment, the subject is diagnosed with RA with a disease duration of less than 1 year.

In another embodiment, the subject has a DAS28 of >3.2.

In another embodiment, the subject has no prior exposure to systemic anti-TNFα therapies, treatment by MTX or >2 DMARDs, and/or has no other acute inflammatory joint diseases.

In another embodiment, the subject is further, e.g., concurrently, administered MTX.

In another embodiment, the subject is administered MTX once weekly, and adalimumab once every 2 weeks.

In one embodiment, the method of the invention includes assaying a sample (or multiple samples from a subject) for multiple genetic markers, including, for example, both the HLA-DRB1 SE allele (e.g., copy number thereof) and the IL-4R I50 allele. Alternatively, the invention includes assaying a sample for the HLA-DRB1 SE allele (e.g., copy number thereof) and the IL-4R V50 allele (e.g., to determine whether subject is homozygous for allele). Furthermore, use of the FcγRIIb I232T single nucleotide polymorphism (SNP) can be used alone or in combination with any of the methods described herein, including the copy number of the HLA-DRB1 SE allele and/or the presence of the IL-4R I50 allele and/or whether the subject is homozygous for the IL-4R V50 allele.

In one embodiment, the TNFα inhibitor is an anti-TNFα antibody, or antigen-binding portion thereof, or a fusion protein, e.g., etanercept.

In one embodiment, the anti-TNFα antibody, or antigen-binding portion thereof, is selected from the group consisting of a human antibody, a chimeric antibody, a humanized antibody, and a multivalent antibody.

In one embodiment, the chimeric anti-TNFα antibody, or antigen-binding portion thereof, is infliximab.

In one embodiment, the human anti-TNFα antibody, or antigen-binding portion thereof, is adalimumab or golimumab.

In one embodiment, the humanized anti-TNFα antibody, or antigen-binding portion thereof, is certolizumab pegol.

In one embodiment, the human anti-TNFα antibody, or antigen-binding portion thereof, is an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

In one embodiment, the human anti-TNFα antibody, or antigen-binding portion thereof, is an isolated human antibody with the following characteristics: dissociates from human TNFα with a k_{off} rate constant of 1×10⁻³ s⁻¹ or less, as determined by surface plasmon resonance; has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In one embodiment, the human anti-TNFα antibody, or antigen-binding portion thereof, is an isolated human antibody with a light chain variable region (LCVR) comprising the amino acid sequence of SEQ ID NO: 1 and a heavy chain variable region (HCVR) comprising the amino acid sequence of SEQ ID NO: 2.

The invention also features a kit for predicting or assessing a subject's responsiveness to a TNFα inhibitor for the treatment of an autoimmune disease, such as rheumatoid arthritis (RA), the kit comprising a means for determining the presence of an HLA-DRB1 SE allele in a sample from the subject, and instructions for recommended treatment for the subject based on the number of copies of the HLA-DRB1 SE allele, wherein the presence of the HLA-DRB1 SE allele indicates that the subject will be responsive to treatment with the TNFα inhibitor.

The presence of the HLA-DRB1 SE allele may be determined according to standard methods known in the art. In one embodiment, the means for determining the number of copies of the HLA-DRB1 SE allele comprises a nucleic acid that hybridizes to HLA-DRB1 SE. In another embodiment, the means for determining the number of copies of the HLA-DRB1 SE allele comprises an antibody which binds to a protein corresponding to HLA-DRB1 SE.

In one embodiment, the lit further comprises a means for detecting the presence of an IL-4R I50 allele in the sample from the subject, and instructions for recommended treatment for the subject based on the presence of the IL-4R I50 allele, wherein the combined presence of the IL-4R I50 allele and the HLA-DRB1 SE allele indicates that the subject will be responsive to treatment of RA with the TNFα inhibitor.

In one aspect, the present invention provides a kit for predicting or assessing a subject's responsiveness to a TNFα inhibitor for the treatment of an autoimmune disease, such as rheumatoid arthritis (RA), the kit comprising a means for determining the presence of an FcγRIIb T232 allele in a sample from the subject, and instructions for recommended treatment for the subject based on the presence of two FcγRIIb T232 alleles (FcγRIIb-CC), wherein the presence of two FcγRIIb T232 alleles indicates the subject will be responsive to treatment with the TNFα inhibitor.

In one embodiment, the means for determining the presence of the FcγRIIb T232 allele comprises a nucleic acid that hybridizes to a nucleic acid molecule encoding FcγRIIb T232, or a portion thereof containing the I232T SNP. In another embodiment, the means for determining the presence of the FcγRIIb T232 allele comprises an antibody which specifically binds to a protein corresponding to an FcγRIIb T232 protein.

In one embodiment, the kit further comprises a means for detecting the presence of an IL-4R I50 allele in the sample from the subject, and instructions for recommended treatment for the subject based on the presence of the IL-4R I50 allele, wherein the combined presence of the IL-4R I50 allele and the FcγRIIb-CC allele indicates that the subject will be responsive to treatment or RA with the TNFα inhibitor. Optionally, the kit further comprises a means for detecting the presence of an HLA-DRB1 SE allele in the sample from the subject, and instructions for recommended treatment for the subject based on the presence of the HLA-DRB1 SE allele, wherein the combined presence of the FcγRIIb-CC allele, the IL-4R I50 allele, and the HLA-DRB1 SE allele indicates that the subject will be responsive to treatment of RA with the TNFα inhibitor.

In yet another embodiment, the kit further comprises means for detecting the presence of an HLA-DRB1 SE allele in the sample from the subject, and instructions for recommended treatment for the subject based on the presence of the HLA-DRB1 SE allele, wherein the combined presence of the FcγRIIb-CC allele and the HLA-DRB1 SE allele indicates that the subject will be responsive to treatment of RA with the TNFα inhibitor.

In one embodiment, the kits further comprise a means for obtaining the sample from the subject.

The invention also provides a kit for predicting or assessing a subject's responsiveness to a TNFα inhibitor for the treatment of an autoimmune disease, such as rheumatoid arthritis (RA), the kit comprising a means for determining the presence of an IL-4R I50 allele in a sample from the subject, and instructions for recommended treatment for the subject based on the presence of the a IL-4R I50 allele, wherein the presence of the IL-4R I50 allele (preferably two copies of the IL-4R I50 allele) in the sample indicates that the subject will be responsive to treatment with the TNFα inhibitor.

In one embodiment, the means for determining the presence of the IL-4R I50 allele comprises a nucleic acid that hybridizes to IL-4R I50. In another embodiment, the means for determining the presence of the IL-4R I50 allele comprises an antibody which binds to a protein corresponding to an IL-4R I50 protein.

The invention further features a kit for predicting or assessing a subject's responsiveness to a TNFα inhibitor for the treatment of an autoimmune disease, such as rheumatoid arthritis (RA), the kit comprising a means for determining the number of copies of a IL-4R V50 allele in a sample from the subject, and instructions for recommended treatment for the subject based on the presence of the a IL-4R V50 allele, wherein two copies of the IL-4R V50 allele in the sample indicates that the subject will not be responsive to treatment with the TNFα inhibitor, unless the subject also has at least one copy of the HLA-DRB1 SE allele.

In one embodiment, the means for determining the presence of the IL-4R V50 allele comprises a nucleic acid that hybridizes to IL-4R V50. In one embodiment, the means for determining the presence of the IL-4R V50 allele comprises an antibody which binds to a protein corresponding to an IL-4R V50 protein.

In one embodiment, the kits of the invention includes means for determining the presence and/or copy number in a sample (or multiple samples from a subject) for multiple genetic markers, including, for example, both the HLA-DRB1 SE allele (e.g., copy number thereof) and the IL-4R I50 allele. Alternatively, the kit includes means for determining the presence and/or copy number of the HLA-DRB1 SE allele (e.g., copy number thereof) and the IL-4R V50 allele. Furthermore, the kits of the invention may include means for determining the presence and/or copy number of the FcγRIIb I232T single nucleotide polymorphism (SNP) alone or in combination with any of the kits described herein, including kits comprising means for determining the presence and/or copy number of the HLA-DRB1 SE allele and/or the presence of the IL-4R I50 allele and/or whether the subject is homozygous for the IL-4R V50 allele.

In one embodiment, the TNFα inhibitor is an anti-TNFα antibody, or antigen-binding portion thereof, or a fusion protein, e.g., etanercept.

In one embodiment, the anti-TNFα antibody, or antigen-binding portion thereof, is selected from the group consisting of a human antibody, a chimeric antibody, a humanized antibody, and a multivalent antibody.

In one embodiment, the chimeric anti-TNFα antibody, or antigen-binding portion thereof, is infliximab.

In one embodiment, the human anti-TNFα antibody, or antigen-binding portion thereof, is adalimumab or golimumab.

In one embodiment, the humanized anti-TNFα antibody, or antigen-binding portion thereof, is certolizumab pegol.

In one embodiment, the human anti-TNFα antibody, or antigen-binding portion thereof, is an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

In one embodiment, the human anti-TNFα antibody, or antigen-binding portion thereof, is an isolated human antibody with the following characteristics: dissociates from human TNFα with a k_{off} rate constant of 1×10⁻³ s⁻¹ or less, as determined by surface plasmon resonance; has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In one embodiment, the human anti-TNFα antibody, or antigen-binding portion thereof, is an isolated human antibody with a light chain variable region (LCVR) comprising the amino acid sequence of SEQ ID NO: 1 and a heavy chain variable region (HCVR) comprising the amino acid sequence of SEQ ID NO: 2.

It is contemplated that all embodiments of the invention described herein, including those described under different aspects of the invention, can be combined with any other embodiments unless inappropriate or explicitly disclaimed.

### BRIEF DESCRIPTION OF THE FIGURES

FIG. 1 shows the design of the OPTIMA study.

FIG. 2 shows the differences in percentage points, by the presence of the HLA-DRB1 SE allele, between treatment groups (adalimumab+methotrexate versus placebo+methotrexate) for subjects who achieved ACR20, ACR50 and ACR70 responses at week 26.

FIG. 3 shows the differences in percentage points, by the presence of the HLA-DRB1 SE allele, between treatment groups (adalimumab+methotrexate versus placebo+methotrexate) for subjects who met DAS28 criteria for LDA and remission at week 26.

FIG. 4 shows the differences in percentage points, by the presence of IL-4 alleles (AA, AG and GG), between treatment groups (adalimumab+methotrexate versus placebo+methotrexate) for subjects who achieved ACR20, ACR50 and ACR70 responses at week 26.

FIG. 5 shows the differences in percentage points, by the presence of IL-4 alleles (AA, AG and GG), between treatment groups (adalimumab+methotrexate versus placebo+methotrexate) for subjects who met DAS28 criteria for LDA and remission at week 26.

FIG. 6 shows a bar graph showing the percentage of adalimumab-treated patients with IL-4R-AA achieving ACR50 and DAS28 at week 26, by SE copy number.

FIG. 7 shows a bar graph depicting the percentage of adalimumab-treated patients with IL-4R-AG achieving ACR50 and DAS28 at week 26, by SE copy number.

FIG. 8 graphically depicts the percentage of adalimumab-treated patients with IL-4R-GG achieving ACR50 and DAS28 at week 26, by SE copy number.

FIG. 9 shows a bar graph describing the percentage of patients with DAS28 low disease activity, by genotype.

### DETAILED DESCRIPTION OF THE INVENTION

### Definitions

In order that the present invention may be more readily understood, certain terms are first defined.

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include, in one embodiment, recombinant human TNFα (rhTNFα), which can be prepared by standard recombinant expression methods or purchased commercially (R & D Systems, Catalog No. 210-TA, Minneapolis, Minn.). TNFα is also referred to as TNF or TNFa.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (REMICADE®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment; certolizumab pegol or CIMZIA®; UCB Group), an anti-TNF dAb (Peptech), CNTO 148 (golimumab; Medarex and Centocor, see WO 02/12502), and adalimumab (HUMIRA®, Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (ENBREL®, Amgen; described in WO 91/03553 and WO 09/406,476, incorporated by reference herein). In another embodiment, the TNFα inhibitor is a recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnnson et al. (1995) J. Mol. Recognit. 8:125; and Johnnson et al. (1991) Anal. Biochem. 198:268.

The term “k_{off},” as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of rheumatoid arthritis).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g., throughout the course of treatment. The biweekly dosing regimen is not intended to include a weekly dosing regimen. In one embodiment, the substance is administered every 9-19 days, every 11-17 days, every 13-15 days, and every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “treatment,” as used within the context of the present invention, is meant to include therapeutic treatment, as well as prophylactic or suppressive measures, for the treatment of rheumatoid arthritis. For example, the term treatment may include administration of a TNFα inhibitor prior to or following the onset of rheumatoid arthritis thereby preventing or removing signs of the disease or disorder. As another example, administration of a TNFα inhibitor after clinical manifestation of rheumatoid arthritis to combat the symptoms and/or complications and disorders associated with rheumatoid arthritis comprises “treatment” of the disease. Further, administration of the agent after onset and after clinical symptoms and/or complications have developed where administration affects clinical parameters of the disease or disorder and perhaps amelioration of the disease, comprises “treatment” of rheumatoid arthritis. In one embodiment, treatment of rheumatoid arthritis in a subject comprises reducing signs and symptoms. In another embodiment, treatment of rheumatoid arthritis in a subject comprises inducing major clinical response of rheumatoid arthritis. In another embodiment, treatment of rheumatoid arthritis in a subject comprises inhibiting the progression of structural damage. In one embodiment, treatment of rheumatoid arthritis comprises improving physical function in adult patients with moderately to severely active disease.

Those “in need of treatment” include mammals, such as humans, already having rheumatoid arthritis, including those in which the disease or disorder is to be prevented.

A TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with rheumatoid arthritis, and related complications and symptoms.

In one embodiment, the TNFα inhibitor used in the invention is an TNFα antibody (also referred to herein as a TNFα antibody), or an antigen-binding fragment thereof, including chimeric, humanized, and human antibodies. Examples of TNFα antibodies which may be used in the invention include, but not limited to, infliximab (REMICADE®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA®, Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors which may be used in the methods and compositions of the invention include etanercept (Enbrel, described in WO 91/03553 and WO 09/406,476), soluble TNF receptor Type I, a pegylated soluble TNF receptor Type I (PEGs TNF-R1), p55 TNFR IgG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFα antibody” excludes adalimumab. In another embodiment, the term “TNFα antibody” excludes adalimumab and infliximab.

As used herein, the term “patient” refers to any single animal, more preferably a mammal (including humans and such non-human animals as, e.g., dogs, cats, horses, rabbits, zoo animals, cows, pigs, sheep, and non-human primates), for which treatment is desired. Most preferably, the patient herein is a human.

As used herein, a “subject” is any single human subject, including a patient, eligible for treatment who is experiencing or has experienced one or more signs, symptoms, or other indicators of RA, whether, for example, newly diagnosed or previously diagnosed and now experiencing a recurrence or relapse, or is at risk for RA, no matter the cause. Intended to be included as a subject are any subjects involved in clinical research trials not showing any clinical sign of disease, or subjects involved in epidemiological studies, or subjects once used as controls. The subject may have been previously treated with a medicament for RA, including a TNFa inhibitor, or not so treated.

A “kit” is any article of manufacture (e.g., a package or container) comprising at least one reagent, e.g., a medicament for treatment of RA, or a probe for specifically detecting a biomarker gene or protein of the invention. The article of manufacture is preferably promoted, distributed, or sold as a unit for performing the methods of the present invention.

The term “sample” shall generally mean any biological sample obtained from an individual, body fluid, body tissue, cell line, tissue culture, or other source. Body fluids are, e.g., lymph, sera, whole fresh blood, peripheral blood mononuclear cells, frozen whole blood, plasma (including fresh or frozen), urine, saliva, semen, synovial fluid, and spinal fluid. Samples also include synovial tissue, skin, hair follicle, and bone marrow. Methods for obtaining tissue biopsies and body fluids from mammals are well known in the art. If the term “sample” is used alone, it shall still mean that the “sample” is a “biological sample”, i.e., the terms are used interchangeably. A “genetic sample” is a sample containing genetic material such as nucleic acids, especially DNA. Typically, the genetic material can be extracted from the sample by conventional means and analyzed for polymorphisms and alleles to determine the presence or expression of biomarkers. Genetic samples include blood and other body fluids as well as tissues and cells.

The verbs “determine” and “assess” shall have the same meaning and are used interchangeably throughout the application.

### Genetic Markers Used in Embodiments of Invention

The invention provides three genetic markers, i.e., HLA-DRB1 SE allele, IL-4R I50V single nucleotide polymorphism (SNP) and/or FcγRIIb I232T SNP (used alone or in combination with one another) for assessing whether a subject having rheumatoid arthritis will be responsive to treatment with a TNFα inhibitor, including, for example, a human anti-TNFa antibody, such as adalimumab.

As used herein, individual amino acids in a sequence are represented as AN or NA or ANA, wherein A is the amino acid in the sequence and N is the position in the sequence. For example, I50V represents a single-amino-acid polymorphism at amino acid position 50, wherein isoleucine (I) is present in the more frequent protein variant in the population and valine (V) is present in the less frequent variant. In another example, I50 represents an isoleucine at position 50.

### HLA-DRB1 Shared Epitope (SE)

The HLA-DRB1 shared epitope (HLA-DRB1 SE) allele is a genetic factor implicated as being responsible for ⅓ of RA susceptibility and in modulating disease activity (see Calin A et al., Arthritis Rheum. 32:1221-5 (1989)). The HLA-DRB1 SE alleles are referred to collectively as “shared epitope” (SE) alleles because of their sequence similarity at positions 70-74 within the third hypervariable region of the HLA-DRB1 alleles (Gregersen et al., Arthritis Rheum., 30:1205-1213 (1987)). The nucleotide and amino acid sequences of HLA-DRB1 are known and can be found in, for example, GenBank Accession Nos. NM_{—}002124.2 and NP_{—}002115, the entire contents of which are incorporated herein by reference. The most highly conserved amino acid sequence within the HLA-DRB1 SE alleles is “RAA” at positions 72-74. In one embodiment, an HLA-DRB1 SE allele suitable for use in the methods and compositions of the invention is one or more of HLA-DRB1 *0101 (QRRAA) (SEQ ID NO:43), *0102 (QRRAA) (SEQ ID NO:44), *0103 (DERAA) (SEQ ID NO:45), *03 (QKRGR) (SEQ ID NO:46), *0401 (QKRAA) (SEQ ID NO:47), *0402 (DERAA) (SEQ ID NO:48), *0403 (QRRAE) (SEQ ID NO:49), *0404 (QRRAA) (SEQ ID NO:50), *0405 (QRRAA) (SEQ ID NO:51), *0407 (QRRAE) (SEQ ID NO:52), *0408 (QRRAA) (SEQ ID NO:53), *0411 (QRRAE) (SEQ ID NO:54), *07, (DRRGQ) (SEQ ID NO:55), *08 (DRRAL) (SEQ ID NO:56), *0901 (RRRAE) (SEQ ID NO:57), *1001 (RRRAA) (SEQ ID NO:58), *1101 (DRRAA) (SEQ ID NO:59), *1102 (DERAA) (SEQ ID NO:60), *1103 (DERAA) (SEQ ID NO:61), *1104 (DRRAA) (SEQ ID NO:62), *12 (DRRAA) (SEQ ID NO:63), *1301 (DERAA) (SEQ ID NO:64); *1302 (DERAA) (SEQ ID NO:65); *1303 (DKRAA) (SEQ ID NO:66), *1323 (DERAA) (SEQ ID NO:67), *1401 (RRRAA) (SEQ ID NO:68), *1402 (QRRAA) (SEQ ID NO:69), *1404 (RRRAE) (SEQ ID NO:70), *15 (QARAA) (SEQ ID NO:71), and *16 (DRRAA) (SEQ ID NO:72) (see, e.g., Essentials of Genomic and Personalized Medicine, eds. G. Ginsberg and H. Willard (2010) Academic Press, San Diego, Calif., p. 553, the contents of which are expressly incorporated herein by reference; the amino acid residues indicated in parentheses following the HLA-DRB1 SE allele correspond to residues 70-74 of NP_{—}002115). In another embodiment, an HLA-DRB1 SE allele suitable for use in the methods of the invention is one or more of *01, e.g., *0101, *0102, and *0103, *04, e.g., *0401, *0402, *0403, *0404, *0405, *0407, *0408, and *0411, *1001, and *14, e.g.,*1401, *1402, and *1404.

### IL-4R

Interleukin 4 receptor (IL-4R) is a multifunctional cytokine that plays a role in the regulation of immune responses (Nelms et al. (1999) Ann Rev Immunol 17:701). The IL-4R I50V (A→G [I50V]) single nucleotide polymorphism (SNP) is a genetic marker associated with early joint erosion (see Prots I. et al., Arthritis Rheum. 54:1491-500 (2006)). “IL-4R I50V single-nucleotide polymorphism” or “IL-4R I50V SNP” as used herein refers to a variation at position 50 of the amino acid sequence of IL-4R. This allelic variation is changing an isoleucine to a valine, which is caused by a variation in the corresponding encoded gene from A to G of the corresponding polynucleotide. The nucleotide and amino acid sequences of IL-4R are known and can be found in, for example, GenBank Accession Nos. NM_{—}000418.2, NM_{—}001008699, NP_{—}000409.1, and NP_{—}001008699.1, the entire contents of which are incorporated herein by reference. The nucleic acid and amino acid sequences of IL-4R can also be found in U.S. Pat. No. 7,205,106, which is incorporated herein by reference. The amino acid and nucleic acid sequences of the mature IL-4R protein are also provided below as SEQ ID Nos: 38 and 39.

MKVLQEPTCVSDYMSISTCEWKMNGPTNCSTELRLLYQLVFLLSEAHTCIPENN GGAGCVCHLLMDDVVSADNYTLDLWAGQQLLWKGSFKPSEHVKPRAPGNLT VHTNVSDTLLLTWSNPYPPDNYLYNHLTYAVNIWSENDPADFRIYNVTYLEPSL RIAAST LKSGISYRAR VRAWAQCYNT TWSEWSPSTK WHNSYREPFE QHLLLGVSVSCIVILAVCLL CYVSITKIKK EWWDQIPNPA RSRLVAIIIQ DAQGSQWEKR SRGQEPAKCPHWKNCLTKLL PCFLEHNMKR DEDPHKAAKE MPFQGSGKSA WCPVEISKTV LWPESISVVRCVELFEAPVE CEEEEEVEEE KGSFCASPES SRDDFQEGRE GIVARLTESL FLDLLGEENGGFCQQDMGES CLLPPSGSTS AHMPWDEFPS AGPKEAPPWG KEQPLHLEPS PPASPTQSPD NLTCTETPLV IAGNPAYRSF SNSLSQSPCP RELGPDPLLARHLEEVEPEM PCVPQLSEPT TVPQPEPETWEQILRRNVLQHGAAAAPVSAPTSGYQEFVH AVEQGGTQASAVVGLGPPGE AGYKAFSSLL ASSAVSPEKC GFGASSGEEG YKPFQDLIPGCPGDPAPVPV PLFTFGLDREPPRSPQSSHL PSSSPEHLGL EPGEKVEDMP KPPLPQEQAT DPLVDSLGSG IVYSALTCHLCGHLKQCHGQ EDGGQTPVMA SPCCGCCCGD RSSPPTTPLR APDPSPGGVP LEASLCPASL APSGISEKSK SSSSFHPAPG NAQSSSQTPK IVNFVSVGPT YMRVS
(SEQ ID NO: 38) (I50V SNP is highlighted in bold/underlined)
atgaaggtcttgcaggagcccacctgcgtctccgactacatgagcatctctacttgcgagtggaagatgaatggtcccaccaatt gcagcaccgagctccgcctgttgtaccagctggtttttctgctctccgaagcccacacgtgtatccctgagaacaacggaggcg cggggtgcgtgtgccacctgctcatggatgacgtggtcagtgcggataactatacactggacctgtgggctgggcagcagctg ctgtggaagggctccttcaagcccagcgagcatgtgaaacccagggccccaggaaacctgacagttcacaccaatgtctcc gacactctgctgctgacctggagcaacccgtatccccctgacaattacctgtataatcatctcacctatgcagtcaacatttggagt gaa aacgacccgg cagatttcag aatctataac gtgacctacctagaaccctc cctccgcatc gcagccagca ccctgaagtc tgggatttcc tacagggcacgggtgagggc ctgggctcag tgctataaca ccacctggag tgagtggagc cccagcacca agtggcacaa ctcctacagg gagcccttcg agcagcacct cctgctgggc gtcagcgtttcctgcattgt catcctggcc gtctgcctgt tgtgctatgt cagcatcacc aagattaagaaagaatggtg ggatcagatt cccaacccag cccgcagccg cctcgtggct ataataatccaggatgctca ggggtcacag tgggagaagc ggtcccgagg ccaggaacca gccaagtgcccacactggaa gaattgtctt accaagctct tgccctgttt tctggagcac aacatgaaaagggatgaaga tcctcacaag gctgccaaag agatgccttt ccagggctct ggaaaatcagcatggtgccc agtggagatc agcaagacag tcctctggcc agagagcatc agcgtggtgcgatgtgtgga gttgtttgag gccccggtgg agtgtgagga ggaggaggag gtagaggaagaaaaagggag cttctgtgca tcgcctgaga gcagcaggga tgacttccag gagggaagggagggcattgt ggcccggcta acagagagcc tgttcctgga cctgctcgga gaggagaatgggggcttttg ccagcaggac atgggggagt catgccttct tccaccttcg ggaagtacga gtgctcacat gccctgggat gagttcccaa gtgcagggcc caaggaggca cctccctggggcaaggagca gcctctccac ctggagccaa gtcctcctgc cagcccgacc cagagtccag acaacctgac ttgcacagag acgcccctcg tcatcgcagg caaccctgct taccgcagct tcagcaactc cctgagccag tcaccgtgtc ccagagagct gggtccagac ccactgctggccagacacct ggaggaagta gaacccgaga tgccctgtgt cccccagctc tctgagccaaccactgtgcc ccaacctgag ccagaaacct gggagcagat cctccgccgaaatgtcctccagcatggggc agctgcagcc cccgtctcgg cccccaccag tggctatcag gagtttgtacatgcggtgga gcagggtggc acccaggcca gtgcggtggt gggcttgggt cccccaggag aggctggtta caaggccttc tcaagcctgc ttgccagcag tgctgtgtcc ccagagaaatgtgggtttgg ggctagcagt ggggaagagg ggtataagcc tttccaagac ctcattcctggctgccctgg ggaccctgcc ccagtccctg tccccttgtt cacctttgga ctggacagggagccacctcg cagtccgcag agctcacatc tcccaagcag ctccccagag cacctgggtctggagccggg ggaaaaggta gaggacatgc caaagccccc acttccccag gagcaggccacagaccccct tgtggacagc ctgggcagtg gcattgtcta ctcagccctt acctgccacctgtgcggcca cctgaaacag tgtcatggcc aggaggatgg tggccagacc cctgtcatgg ccagtccttg ctgtggctgc tgctgtggag acaggtcctc gccccctaca acccccctgagggccccaga cccctctcca ggtggggttc cactggaggc cagtctgtgt ccggcctccc tggcaccctc gggcatctca gagaagagta aatcctcatc atccttccat cctgcccctggcaatgctca gagctcaagc cagaccccca aaatcgtgaa ctttgtctcc gtgggacccacatacatgag ggtctct (SEQ ID NO: 39) (I50V SNP is highlighted in bold/underlined)

### FcγRIIb

Fcγ receptor IIb (FcγRIIb; also referred to as CD32 or FCGR2B) is involved in the phagocytosis of immune complexes and in the regulation of antibody production by B-cells. The FcγRIIb I232T (T→C [I232T]) SNP is associated with rapid radiologic joint damage in patients with definite erosive disease, as well as other diseases such as lupus (see Radstake et al. Arthritis Rheum. 54:3828-37 (2006); Kono et al. (2005) Hum Mol Genetic 14:2881). “FcγRIIb I232T single-nucleotide polymorphism” or “FcγRIIb I232T SNP” as used herein refers to a variation at position 232 of the amino acid sequence of FcγRIIb. This allelic variation is changing an isoleucine to a threonine, which is caused by a variation in the corresponding encoded gene from T to C of the corresponding polynucleotide. The nucleotide and amino acid sequences of FcγRIIb are known and can be found in, for example, GenBank Accession Nos. NM_{—}001002273.2, NM_{—}001002274.2, NM_{—}001002275.2, NM_{—}001190828.1, NM_{—}004001.4, NP_{—}001002273.1, NP_{—}001002274.1, NP_{—}001002275.1, NP_{—}001177757.1, NP_{—}003992.3, the entire contents of which are incorporated herein by reference. Exemplary amino acid and nucleotide sequences of FcγRIIb are provided below as SEQ ID Nos: 40 and 41, respectively.

MGILSFLPVLATESDWADCKSPQPWGHMLLWTAVLFLAPVAGTPAAPPKA VLKLEPQWINVLQEDSVTLTCRGTHSPESDSIQWFHNGNLIPTHTQPSYR FKANNNDSGEYTCQTGQTSLSDPVHLTVLSEWLVLQTPHLEFQEGETIVL RCHSWKDKPLVKVTFFQNGKSKKFSRSDPNFSIPQANHSHSGDYHCTGNI GYTLYSSKPVTITVQAPSSS PMGIIVAVVTGIAVAAIVAAVVALIYCRKK RISANPTNPDEADKVGAENT ITYSLLMHPDALEEPDDQNRI
(SEQ ID NO: 40) (I232T SNP is highlighted in bold/underlined; Accession No. NP_{—}001002274 without signal sequence)
atg ggaatcctgt cattcttacc tgtccttgcc actgagagtg actgggctgactgcaagtcc ccccagcctt ggggtcatat gcttctgtgg acagctgtgc tattcctggctcctgttgct gggacacctg cagctccccc aaaggctgtg ctgaaactcg agccccagtggatcaacgtg ctccaggagg actctgtgac tctgacatgc cgggggactc acagccctgagagcgactcc attcagtggt tccacaatgg gaatctcatt cccacccaca cgcagcccagctacaggttc aaggccaaca acaatgacag cggggagtac acgtgccaga ctggccagaccagcctcagc gaccctgtgc atctgactgt gctttctgag tggctggtgc tccagacccctcacctggag ttccaggagg gagaaaccat cgtgctgagg tgccacagct ggaaggacaagcctctggtc aaggtcacat tcttccagaa tggaaaatcc aagaaatttt cccgttcggatcccaacttc tccatcccac aagcaaacca cagtcacagt ggtgattacc actgcacaggaaacataggc tacacgctgt actcatccaa gcctgtgacc atcactgtcc aagctcccagctcttcaccg atggggatca ttgtggctgt ggtcactggg attgctgtag cggccattgttgctgctgta gtggccttga tctactgcag gaaaaagcgg atttcagcca atcccactaatcctgatgag gctgacaaag ttggggctga gaacacaatc acctattcac ttctcatgcacccggatgct ctggaagagc ctgatgacca gaaccgtatt tag
(SEQ ID NO: 41) (I232T SNP is highlighted in bold/underlined; Accession No. NM_{—}001002274)

### Diagnostics

In one aspect, the present invention provides methods for predicting or assessing responsiveness of a subject having or prone to having rheumatoid arthritis, to an anti-TNFα inhibitor. The methods generally include determining the presence or absence of (e.g., the copy number of) an HLA-DRB1 SE, IL-4R I50V SNP and/or FcγRIIb I232T SNP in a biological sample obtained from the subject, wherein the presence of particular allele(s) in the sample is an indication that the subject will respond to treatment with the TNFa inhibitor.

In one embodiment, using the methods described herein and known in the art, a sample from a subject may be tested for the presence of one or both alleles associated with a SNP. For example, a sample of a subject may be tested for the presence of the IL-4R I50 allele (or, alternatively, the IL-4R V50 allele) to determine whether the subject has an AA (I50I), AG (I50V), or GG (V50V) genotype, and, therefore, whether the subject will be responsive to treatment with a TNFa inhibitor. Similarly, a sample of a subject may be tested for the presence of the FcγRIIb I232 allele (or, alternatively, the FcγRIIb T232 allele) to determine whether the subject has an TT (I232I), TC (I232T), or CC (T232T) genotype, and, therefore, whether the subject will be responsive to treatment with a TNFa inhibitor. Detection of a SNP, as described below, refers to determining which allele(s) a subject has.

In one embodiment, using the methods described herein and known in the art, a sample from a subject may be tested for the presence of an HLA-DRB1 SE allele. For example, a sample from a subject may be tested for the presence of the SE region of the HLA-DRB1, e.g., in DNA or protein. It should be noted that the sample can also be tested for the absence of the HLA-DRB1 SE allele, equivalent to an SE allele count of 0.

Detection of the HLA-DRB1 SE allele, IL-4R I50V SNP and/or FcγRIIb I232T SNP may be accomplished using methods described herein and/or using any of the commercially available kits and/or techniques well known in the art. For example, as described in the appended example, high-resolution typing with Protrans S4 Sequencing Kits (Medipro) may be used to determine whether a patient has HLA-DRB1 SE homozygosity or heterozygosity, allele-specific PCR using Assay-on-Demand (Applied Biosystems) was used to determine IL-4R (A to G [I50V]) SNP, and allele-specific PCR using Assay-by-Design (Applied Biosystems) was used to determine FcγRIIb (T to C [I232T]) variant.

Methods for detecting the genetic markers (SE and/or polymorphisms) include protocols that examine the presence and/or expression of the SNP or SE in a sample from a subject. Determining the presence or absence of an HLA-DRB1 SE allele, IL-4R I50 and/or V50 allele (thus distinguishing the I50V polymorphism), and/or FcγRIIb I232 allele and/or T232 allele (thus distinguishing the I232T polymorphism) in the biological sample may also be accomplished using any other well known techniques such as polymerase chain reaction (PCR) amplification reaction, reverse-transcriptase PCR analysis, single-strand conformation polymorphism analysis (SSCP), mismatch cleavage detection, heteroduplex analysis, Southern blot analysis, Western blot analysis, deoxyribonucleic acid sequencing, restriction fragment length polymorphism analysis, haplotype analysis, serotyping, and combinations or sub-combinations thereof.

For example, such samples, including tissue or cell samples, can be conveniently assayed for, e.g., genetic marker mRNAs or DNAs using, for example, a Northern blot method, a Southern blot method, a dot-blot, PCR analysis, array hybridization, RNase protection assay, a FISH method, a CGH method, an RNA chip method, or a DNA chip method, such as a DNA SNP chip microarray (e.g., Affymetrix's microarray system or Illumina's BeadArray Technology). DNA SNP chip microarrays are commercially available, including DNA microarray snapshots. In one embodiment, the methods and kits of the invention are practiced using microarray analysis. In one embodiment, the methods of the invention are performed using a genechip or DNA microarray comprising nucleic acid probes specific for HLA-DRB1 SE, FcγRIIb I232T SNP, and /or IL-4R I50V SNP.

For example, an mRNA sample may be obtained from the subject (e.g., isolated from peripheral blood mononuclear cells, by standard methods) and expression of mRNA(s) encoding an HLA-DRB1 SE allele, IL-4R I50 and/or V50 allele and/or FcγRIIb I232 and/or T232 allele in the mRNA sample may be detected using standard molecular biology techniques, such as PCR analysis. A preferred method of PCR analysis is reverse transcriptase-polymerase chain reaction (RT-PCR). Other suitable systems for mRNA sample analysis include microarray analysis (e.g., using Affymetrix's microarray system or Illumina's BeadArray Technology).

For example, real-time PCR (RT-PCR) assays such as quantitative PCR assays may be also be used to detect the presence or absence of the biomarkers described herein, and such methods are well known in the art. In an illustrative embodiment of the invention, a method for detecting a FcγRIIb I232T SNP mRNA in a biological sample comprises producing cDNA from the sample by reverse transcription using at least one primer; amplifying the cDNA so produced using FcγRIIb I232T SNP polynucleotides as sense and antisense primers to amplify FcγRIIb I232T SNP cDNAs therein; and detecting the presence of the amplified FcγRIIb I232T SNP cDNA. In addition, such methods can include one or more steps that allow one to determine the levels of FcγRIIb I232T SNP mRNA in a biological sample (e.g., by simultaneously examining the levels of a comparative control mRNA sequence of a “housekeeping” gene such as an actin family member). Optionally, the sequence of the amplified FcγRIIb I232T SNP cDNA can be determined.

In one specific embodiment, genotyping of the IL-4RI50V or FcγRIIb I232T polymorphism can be performed by RT-PCR technology, using the TAQMAN™ 5′-allele discrimination assay, a restriction fragment-length polymorphism PCR-based analysis, or a PYROSEQUENCER™ instrument. In addition, the method of detecting a genetic variation or a polymorphism set forth in U.S. Pat. No. 7,175,985, incorporated by reference, may be used. In this method a nucleic acid is synthesized utilizing the hybridized 3′-end, which is synthesized by complementary-strand synthesis, on a specific region of a target nucleotide sequence existing as the nucleotide sequence of the same strand as the origin for the next round of complementary-strand synthesis.

Probes used for PCR may be labeled with a detectable marker, such as, for example, a radioisotope, fluorescent compound, bioluminescent compound, chemiluminescent compound, metal chelator, or enzyme. Such probes and primers can be used to detect the presence of SNP or SE polynucleotides in a sample and as a means for detecting a cell expressing SE or SNP proteins. As will be understood by the skilled artisan, a great many different primers and probes may be prepared based on the sequences provided herein and used effectively to amplify, clone, and/or determine the presence and/or levels of SNP or SE mRNAs.

Any of the genetic markers of the invention, or portions, thereof may also be sequenced to determine the presence or absence in a sample of the SNP or SE. Any of the well-known methods for sequencing one or both strands of the HLA-DRB1 SE allele, IL-4R I50 and/or V50 allele and/or FcγRIIb I232 and/or T232 allele may be used in the methods of the invention, such as the methods described in, for example, U.S. Pat. No. 5,075,216, Engelke et al. (1988) Proc. Natl. Acad. Sci. U.S.A. 85, 544-548 and Wong et al. (1987) Nature 330, 384-386; Maxim and Gilbert (1977) Proc. Natl. Acad. Sci. U.S.A. 74:560; or Sanger (1977) Proc. Natl. Acad. Sci. U.S.A. 74:5463. In addition, any of a variety of automated sequencing procedures can be utilized. See, e.g., Naeve, C. W. et al. (1995) Biotechniques 19:448, including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159. In one embodiment, the HLA-DRB1 SE allele from a sample of a subject is directly sequenced to determine whether the subject has at least one copy of the HLA-DRB1 SE allele.

As indicated above, determining the presence or absence of an HLA-DRB1 SE allele, an IL-4R I50 allele and/or an IL-4R V50 allele, and/or ab FcγRIIb I232 or T232 allele, may include, for example, restriction fragment length polymorphism analysis. Restriction fragment length polymorphism analysis (RFLPS) is based on changes at a restriction enzyme site. Moreover, the use of sequence specific ribozymes (see, for example, U.S. Pat. No. 5,498,531) may be used to score for the presence of a specific ribozyme cleavage site.

Another technique for determining the presence or absence of an HLA-DRB1 SE allele, IL-4R I50 allele V50 allele and/or FcγRIIb I232 allele or T232 allele involves hybridizing DNA segments which are being analyzed (target DNA) with a complimentary, labeled oligonucleotide probe as described in, for example, Wallace et al. (1981) Nucl. Acids Res. 9, 879-894. Since DNA duplexes containing even a single base pair mismatch exhibit high thermal instability, the differential melting temperature may be used to distinguish target DNAs that are perfectly complimentary to the probe from target DNAs that only differ by a single nucleotide. This method has been adapted to detect the presence or absence of a specific restriction site, as described in, for example, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for determining the presence or absence of an HLA-DRB1 SE allele, IL-4R I50 and/or V50 alleles, and/or FcγRIIb I232 allele and/or T232 allele include methods in which protection from cleavage agents is used to detect mismatched bases in RNA/RNA or RNA/DNA heteroduplexes (as described in, for example, Myers et al. (1985) Science 230:1242). In general, the art technique of “mismatch cleavage” starts by providing heteroduplexes of formed by hybridizing (labeled) RNA or DNA containing the polymorphic sequence with potentially polymorphic RNA or DNA obtained from a sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In another embodiment, alterations in electrophoretic mobility may be used to determine the presence or absence of an HLA-DRB1 SE allele, IL-4R I50 and/or V50 allele and/or FcγRIIb I232 and/or T232 allele. For example, single strand conformation polymorphism (SSCP) may be used to detect differences in electrophoretic mobility between various HLA-DRB1 SE, IL-4R I50V and/or FcγRIIb I232T alleles (as described in, for example, Orita et al. (1989) Proc Natl. Acad. Sci. USA: 86:276; Cotton (1993) Mutat Res 285:125-144; and Hayashi (1992) Genet Anal Tech Appl 9:73-79). Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of a nucleic acid molecule in polyacrylamide gels containing a gradient of denaturant is assayed using denaturing gradient gel electrophoresis (DGGE) (as described in, for example, Myers et al. (1985) Nature 313:495. When DGGE is used as the method of analysis, DNA can be modified to ensure that it does not completely denature, for example by adding a GC clamp of approximately 40 bp, of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for determining the presence or absence of an HLA-DRB1 SE allele, IL-4R I50V SNP and/or FcγRIIb I232T SNP include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different polymorphisms when the oligonucleotides are attached to the hybridizing membrane and hybridized with labeled target DNA.

Another process for determining the presence or absence of an HLA-DRB1 SE allele, IL-4R I50V SNP and/or FcγRIIb I232T SNP is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Moreover, any of the well-known methods for genotyping such SNPs (e.g., DNA sequencing, hybridization techniques, PCR based assays, fluorescent dye and quenching agent-based PCR assay (Taqman PCR detection system), RFLP-based techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing, protein truncation assay (PTT), immunoassays, haplotype analysis, and solid phase hydridization (dot blot, reverse dot blot, chips) are very well known in the art and described in, for example, Siitari, Nucleic acid diagnostics market, Technology Review 125/2002, ISDN 1239-758; Caplin (1999) Biochemica 1:5-8; Neville, (2002) BioTechniques 32:34-43; Underhill (197) Genome Res 7:996-1005; Oefner (2000) J Chromatogr B Biomed Sci Appl 739:345-55, and the patent publication No. U.S. 20010049586 and may be used in the methods of the invention.

Yet another suitable method for determining the presence or absence of an HLA-DRB1 SE allele, IL-4R I50V SNP and/or FcγRIIb I232T SNP is serotyping of biological samples from a subject using, e.g., commercially available antibodies for an HLA-DRB1 SE allele, IL-4R I50V SNP and/or FcγRIIb I232T SNP in an ELISA assay.

In certain situations samples may be assayed for the expression of an HLA-DRB1 SE allele, IL-4R I50 and/or V50 allele and/or FcγRIIb I232 and/or T232 allele at the protein level, using a detection reagent that detects the protein product encoded by the mRNA of the marker. For example, if an antibody reagent is available that binds specifically to the protein product of the HLA-DRB1 SE allele, the IL-4R I50 allele or the IL-4R V50 allele, and/or the FcγRIIb I232 or T232 allele to be detected, and not to other proteins, then such an antibody reagent can be used to detect the expression of the HLA-DRB1 SE allele, IL-4R I50 allele or V50 allele and/or FcγRIIb I232 or T232 allele in a sample from the subject, or a preparation derived from the sample, using standard antibody-based techniques known in the art, such as FACS analysis, ELISA and the like. In one embodiment, the antibody can distinguish between the two protein products of the I50 IL-4R allele and the V50 IL-4R allele. In another embodiment, the antibody can distinguish between the two protein products of the FcγRIIb I232 allele and the FcγRIIb T232 allele. In one embodiment, the antibody used in the detection method can identify amino acids 70-74 of HLA-DB1 protein, and, in a further embodiment, is specific for the SE.

Any sample obtained from a subject having or prone to having rheumatoid arthritis may be used to determine the presence or absence of an HLA-DRB1 SE allele, IL-4R I50V SNP and/or FcγRIIb I232T SNP. For example, the sample may be any fluid or sub-component thereof, e.g., blood fluids, vomit, intra-articular fluid, saliva, lymph, cystic fluid, urine, fluids collected by bronchial lavage, fluids collected by peritoneal rinsing, or gynecological fluids, obtained from the subject. In a typical situation, the fluid may be a blood sample, or a component thereof, obtained from the subject. The sample may also be any tissue or fragment or sub-component thereof, e.g., bone, connective tissue, cartilage, lung, liver, kidney, muscle tissue, heart, pancreas, and skin, obtained from the subject.

Techniques or methods for obtaining samples from a subject are well known in the art and include, for example, obtaining samples by a mouth swab or a mouth wash; drawing blood; or obtaining a biopsy. Isolating sub-components of fluid or tissue samples (e.g., cells or RNA or DNA) may be accomplished using well known techniques in the art and those described in the Examples section below.

In another aspect, the invention pertains to a method for predicting or assessing responsiveness of a subject having or prone to having rheumatoid arthritis, to an TNFα inhibitor by contacting a biological sample derived from the subject with an agent capable of detecting the presence or absence of an HLA-DRB1 SE allele, IL-4R I50V SNP and/or FcγRIIb I232T SNP in the sample, wherein the presence of the HLA-DRB1 SE allele and/or the IL-4R I50 allele and/or an FcγRIIb-CC allele (T232T) in the sample is an indication that the subject will respond to the TNFα inhibitor, thereby predicting or assessing responsiveness of the subject to the TNFα inhibitor. By contacting a biological sample derived from the subject with an agent capable of detecting the presence or absence of an HLA-DRB1 SE allele, IL-4R I50V SNP and/or FcγRIIb I232T SNP in the sample, the sample is necessarily transformed or changed in some way from its original form such that detection of the presence or absence of an HLA-DRB1 SE allele, IL-4R I50V SNP and/or FcγRIIb I232T SNP in the sample can be achieved. The agent with which the biological sample is contacted may be, for example, a PCR/sequencing primer(s), nucleotides and enzymes suitable for amplifying and/or sequencing and/or labeling the HLA-DRB1 SE allele, IL-4R I50 or V50 allele and/or FcγRIIb I232 or T232 allele (e.g., a distinct region within HLA-DRB1 SE allele (e.g., nucleic acid sequence corresponding to amino acids 70-74), IL-4R I50 or V50 allele (i.e., region that distinguishes the SNP) and/or FcγRIIb I232 or T232 alleles) present in the sample, an antibody capable of detecting an HLA-DRB1 SE allele, distinguishing IL-4R I50V SNP and/or distinguishing FcγRIIb I232T SNP in the sample, a restriction enzyme, and/or a microarray.

Measurement of biomarker expression levels or presence may be performed by using a software program executed by a suitable processor. Suitable software and processors are well known in the art and are commercially available. The program may be embodied in software stored on a tangible medium such as a CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement of the expression levels or presence of the genes identified herein, or their expression products, and the determination that a subject is likely or not likely to respond to treatment with a TNFa inhibitor, the assay results, findings, diagnoses, predictions, and/or treatment recommendations may be recorded and communicated to technicians, physicians, and/or patients, for example. In certain embodiments, computers will be used to communicate such information to interested parties, such as patients and/or the attending physicians. In some embodiments, the assays will be performed or the assay results analyzed in a country or jurisdiction that differs from the country or jurisdiction to which the results or diagnoses are communicated.

In a preferred embodiment, a diagnosis, prediction, and/or treatment recommendation based on the expression level or presence of a genetic marker in a test subject of one or more of the genetic markers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as e-mail, or telephone. Communication may be facilitated by use of a computer, such as in the case of e-mail communications. In certain embodiments, the communication containing results of a diagnostic test and/or conclusions drawn from and/or treatment recommendations based on the test may be generated and delivered automatically to the subject using a combination of computer hardware and software that will be familiar to artisans skilled in telecommunications. One example of a healthcare-oriented communications system is described in U.S. Pat. No. 6,283,761; however, the present invention is not limited to methods that utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

Selection and Use of Treatment Regimens with TNFα Inhibitors

Given the observation that the presence or absence of an HLA-DRB1 SE allele, IL-4R I50 and/or V50 alleles, and/or FcγRIIb I232 and/or T232 alleles in a subject having or prone to having rheumatoid arthritis (RA) influences the responsiveness of the subject to treatment with a TNFα inhibitor, e.g., a human TNFa antibody, or antigen binding portion thereof, such as, but not limited to, adalimumab, one can select an appropriate treatment regimen for the subject based on the presence or absence of an HLA-DRB1 SE allele, IL-4R I50 and/or V50 alleles, and/or FcγRIIb I232 and/or T232 alleles in the subject. Accordingly, in one embodiment, the invention provides a method for selecting a treatment regimen with the TNFα inhibitor based upon the presence or absence of an HLA-DRB1 SE allele, IL-4R I50 and/or V50 alleles, and/or FcγRIIb I232 and/or T232 alleles in the subject. In another aspect, the method further comprises administering the TNFα inhibitor to the subject according to the treatment regimen such that the rheumatoid arthritis is treated in the subject. In another aspect, the method yet still further comprises administering both MTX and the TNFα inhibitor to the subject according to the treatment regimen such that the rheumatoid arthritis is treated in the subject.

In one aspect, the invention provides a method for selecting a treatment regimen for therapy with an TNFα inhibitor, e.g., a human TNFa antibody, or antigen binding portion thereof, such as, but not limited to, adalimumab, in a subject having or prone to having rheumatoid arthritis. The method include determining the presence or absence (or number of copies) of an HLA-DRB1 SE allele, IL-4R I50 and/or V50 alleles, and/or FcγRIIb I232 and/or T232 alleles in the subject; and selecting a treatment regimen with TNFα inhibitor based upon the presence or absence (or number of copies) of an HLA-DRB1 SE allele, IL-4R I50 and/or V50 alleles, and/or FcγRIIb I232 and/or T232 alleles in the subject.

In one aspect, the invention provides a method of predicting the responsiveness of a subject having rheumatoid arthritis (RA) to treatment with a TNFα inhibitor, e.g., a human TNFa antibody, or antigen binding portion thereof, such as, but not limited to, adalimumab, by determining whether the subject has an HLA-DRB1 SE allele. In one aspect, a sample is obtained from the subject and assessed for the presence (or absence/or number of copies) an HLA-DRB1 SE allele. In another aspect, the invention provides a method of treating a subject having RA by administering a TNFa inhibitor, provided that at least one copy of an HLA-DRB1 shared epitope (HLA-DRB1 SE) allele is present in a sample from the subject. In one embodiment, a sample from the subject is tested for the presence of at least one copy of an HLA-DRB1 shared epitope (HLA-DRB1 SE) allele. As described in the examples below, the presence of at least one copy of the HLA-DRB1 SE allele indicates that the subject will be responsive to treatment with the TNFα inhibitor.

In one aspect, the invention provides a method of predicting the responsiveness of a subject having rheumatoid arthritis (RA) to treatment with a TNFα inhibitor, e.g., a human TNFa antibody, or antigen binding portion thereof, such as, but not limited to, adalimumab, by determining whether the subject has an FcγRIIb T232 allele (or, alternatively whether the subject has an FcγRIIb I232 allele). In one aspect, a sample is obtained from the subject and assessed for the presence (or absence/or number of copies) an FcγRIIb T232 allele (or, alternatively whether the subject has an FcγRIIb I232 allele). In another aspect, the invention provides a method of treating a subject having RA by administering a TNFα inhibitor, provided that two copies of an FcγRIIb T232 allele are present in a sample from the subject. As described in the examples below, the presence of FcγRIIb-CC allele indicates that the subject will be responsive to treatment with the TNFα inhibitor.

In one embodiment of the invention, the presence (or absence/or number of copies) of an HLA-DRB1 SE allele is tested in combination with the presence of FcγRIIb I232 and/or a FcγRIIb T232 allele to determine whether a subject having RA will be responsive to treatment with a TNFa inhibitor, e.g., a human TNFa antibody, or antigen binding portion thereof, such as, but not limited to, adalimumab. In one embodiment of the invention, the presence of an HLA-DRB1 SE allele is tested in combination with the presence of IL-4R I50 and/or IL-4R V50 allele to determine whether a subject having RA will be responsive to treatment with a TNFa inhibitor. In one embodiment of the invention, the presence of an HLA-DRB1 SE allele is tested in combination with the presence of an IL-4R I50 and/or IL-4R V50 allele, and an FcγRIIb I232 and/or a FcγRIIb T232 allele to determine whether a subject having RA will be responsive to treatment with a TNFa inhibitor. In one embodiment, the presence of an FcγRIIb I232 and/or a FcγRIIb T232 allele is tested in combination with the presence of an IL-4R I50 and/or IL-4R V50 allele to determine whether a subject having RA will be responsive to treatment with a TNFa inhibitor.

In one embodiment, the genetic markers described herein may be used in a method of selecting a patient having RA who will be responsive to treatment with a TNFa inhibitor, e.g., a human TNFa antibody, or antigen binding portion thereof, such as, but not limited to, adalimumab.

In one embodiment, in determining the presence of an allele, the method may include determining the number of copies of the allele. Alternatively, the assay method may just determine the presence or absence of the genetic marker.

In another embodiment, the invention also provides a method of treating a subject having rheumatoid arthritis with an TNFα inhibitor. The method includes determining the presence or absence of an HLA-DRB1 SE allele, IL-4R I50 and/or V50 alleles, and/or FcγRIIb I232 and/or T232 alleles in the subject, selecting a treatment regimen with an TNFα inhibitor based upon the presence or absence of an HLA-DRB1 SE allele, IL-4R I50 and/or Il-4R V50 alleles, and/or FcγRIIb I232 and/or T232 alleles in the subject, and administering the TNFα inhibitor according to the treatment regimen such that the subject is treated for the rheumatoid arthritis.

The treatment regimen that is selected typically includes at least one of the following parameters and may include many or all of the following parameters: the type of agent chosen for administration, the dosage, the formulation, the route of administration and/or the frequency of administration.

As described in the examples below, including, for example, in subjects with at least 1 copy of the HLA-DRB1 SE allele or the FcγRIIb-CC allele, combination therapy with adalimumab and methotrexate was associated with significantly improved clinical responses compared with methotrexate monotherapy. In addition, significantly enhanced clinical response was observed for patients on adalimumab and methotrexate who were either homozygous or heterozygous for the IL-4R I50 alleles (AA or AG) but not in patients with two IL-4R V50 alleles (GG). FcγRIIb-CC was significantly associated with achieving clinical responses. Furthermore, in combination, the effect of SE copy number was muted in the IL-4R-AA and FcγRIIb-TT wild type backgrounds, but apparent when at least 1 copy of either the IL-4R (AG or GG) or FcγRIIb (TC or CC) genetic variants were present.

Methods of treatment described herein may include administration of a TNFα inhibitor to a subject to achieve a therapeutic goal, e.g., achieving a certain ACR response, e.g., ACR20, ACR50, ACR70 and/or improving DAS28 score, including, for example, DAS28 low disease activity (DAS28 LDA) or DAS28 remission.

DAS28 (disease activity score) is known in the art as an accepted measure of the activity of rheumatoid arthritis in an affected subject. The following parameters are included in the calculation: Number of joints tender to the touch (TEN); Number of swollen joints (SW); Erythrocyte sedimentation rate (ESR); Patient assessment of disease activity (VAS; mm) (see Van der Heijde et al. Ann Rheum Dis 1990; 49:916-20). In modified DAS (DAS28) 28 joints are assessed (see Prevoo M L L, et al. Arthritis Rheum 1995; 38:44-8).

The American College of Rheumatology preliminary criteria for improvement in Rheumatoid Arthritis (ACR20, 50, 70 responses) was developed to provide a efficacy measures for rheumatoid arthritis (RA) treatments. ACR20, ACR50 and ACR70 requires a greater than 20%, 50% and 70% improvement respectively. Response criteria are detailed in Felson D T, Anderson J J, Boers M, Bombardier C, Furst D, Goldsmith C, et al. American College of Rheumatology preliminary definition of improvement in rheumatoid arthritis. Arthritis Rheum 1995; 38:727-35, incorporated by reference herein. Generally, patients are examined clinically at screening, baseline, and frequently during treatment. The primary efficacy for signs and symptoms is measured via American College of Rheumatology preliminary criteria for improvement (ACR20) at 12 weeks. An additional primary endpoint includes evaluation of radiologic changes over 6 to 12 months to assess changes in structural damage.

In one embodiment, the subject is treated with a TNFα inhibitor in accordance with a biweekly dosing regimen. Biweekly dosing regimens are further described in U.S. application Ser. No. 10/163,657 (US 20030235585), incorporated by reference herein.

In one aspect of the invention, the TNFα inhibitor is administered to the subject having RA as a fixed dose (in contrast to a mg/kg dose). In one embodiment, the fixed dose is about 20-80 mg, about 20-60 mg, about 30-50 mg, or about 40 mg. In a further embodiment, the fixed dose is about 50 mg.

In one embodiment, the subject is subcutaneously administered 40 mg of a human TNFα antibody, or antigen binding portion thereof, every other week for the treatment of RA.

In another aspect of the invention, the subject is treated with a TNFα inhibitor in accordance with a monthly dosing regimen. In one embodiment, the subject is subcutaneously administered 50 mg of a human TNFα antibody, or antigen binding portion thereof, once a month for the treatment of RA.

In a further embodiment, the TNFα inhibitor is administered to the subject in combination with methotrexate for the treatment of RA.

### TNFα Inhibitors of Invention

Particularly preferred TNFα inhibitors are biologic agents that have been approved by the FDA for use in humans in the treatment of rheumatoid arthritis or are undergoing clinical testing for the treatment of rheumatoid arthritis.

In one embodiment, the invention features uses and composition for predicting or determining the efficacy of a TNFα inhibitor for the treatment of rheumatoid arthritis, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2; the nucleic acid sequence of the VL and VH domains are described in SEQ ID Nos: 36 and 37, respectively). The properties of D2E7 (adalimumab/HUMIRA®) have been described in Salfeld et al., U.S. Pat. Nos. 6,090,382, 6,258,562, and 6,509,015, which are each incorporated by reference herein.

In one embodiment, the TNFa inhibitor is a fully human TNFa antibody which is a biosimilar to adalimumab. In one embodiment, the TNFa inhibitor is highly similar to adalimumab, and may, for example, include minor differences in clinically inactive components. In one embodiment, the TNFa inhibitor is interchangeable with adalimumab, and is, for example, able to produce the same clinical result as adalimumab in any given patient.

In one embodiment, the method of the invention includes determining the efficacy of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of rheumatoid arthritis. In one embodiment, the invention provides treatment with an isolated human antibody, or an antigen-binding portion thereof, that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a k_{off} of 5×10⁻⁴ s⁻¹ or less, or even more preferably, with a k_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating Crohn's disease by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the k_{off}. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the k_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the k_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 are critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a k_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a k_{off} of 5×10⁻⁴ s⁻¹ or less. Even more preferably, the antibody, or antigen-binding portion thereof, dissociates from human TNFα with a k_{off} of 1×10⁻⁴ s⁻¹ or less.

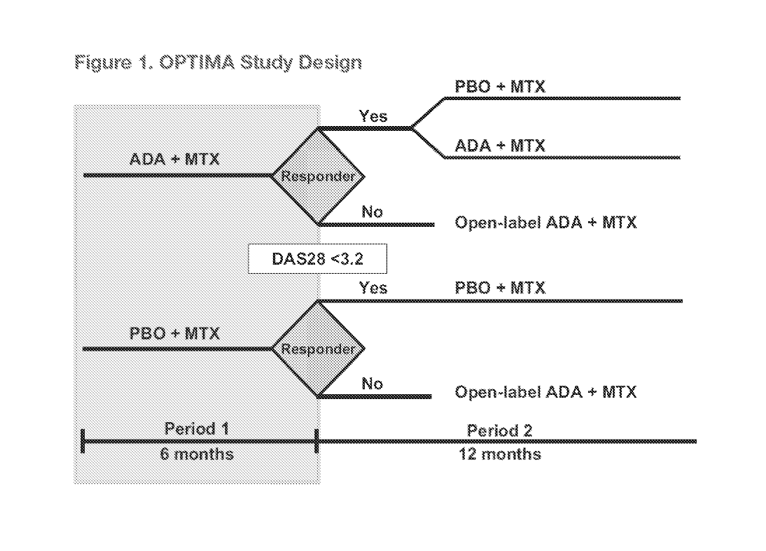

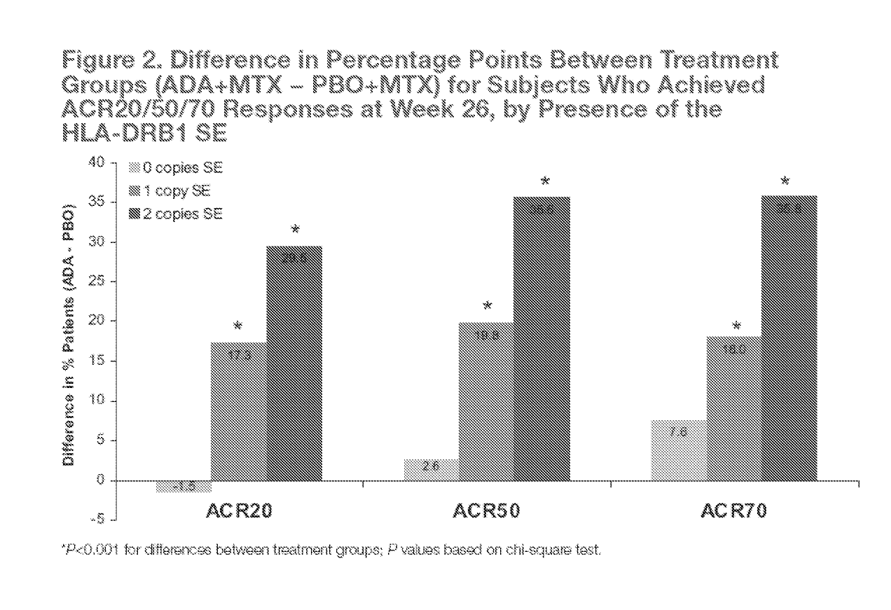

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. In one embodiment, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). In one embodiment, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{K}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342). I

The TNFα antibody used in the methods and compositions of the invention may be modified for improved treatment of rheumatoid arthritis. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat rheumatoid arthritis by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by cross-linking two or more antibodies (of the same type or of different types, e.g., to create bispecific antibodies). Suitable cross-linkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the V_{H}3 family of human germline VH genes is amplified by standard PCR. Most preferably, the DP-31 VH germline sequence is amplified. To obtain a DNA fragment encoding the light chain variable region of D2E7, or a D2E7-related antibody, a member of the V_{K}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃ (SEQ ID NO:42) such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13). Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat rheumatoid arthritis.

In one embodiment, the antibody or antibody portion for use in the methods of the invention is incorporated into a pharmaceutical formulation as described in PCT/IB03/04502 and U.S. Appln. No. 20040033228, incorporated by reference herein. This formulation includes a concentration 50 mg/ml of the antibody D2E7 (adalimumab), wherein one pre-filled syringe contains 40 mg of antibody for subcutaneous injection. Alternative formulations containing high concentrations of adalimumab are described in both US20090291062 and US20100278822, the contents of each of which are incorporated by reference herein

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

The TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including a rheumatoid arthritis inhibitor or antagonist. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### Kits of Invention

The invention also provides kits for assessing a subject's responsiveness to a TNFα inhibitor for the treatment of rheumatoid arthritis (RA), a well as kits for treating a subject having rheumatoid arthritis (RA). These kits include means for determining the number of copies (or presence or absence) of an HLA-DRB1 SE, IL-4R I50 and/or V50 allele, and/or FcγRIIb I232 and/or T232 allele and instructions for use of the kit.

The kits of the invention may optionally comprise additional components useful for performing the methods of the invention. By way of example, the kits may comprise means for obtaining a biological sample from a subject, a control sample, e.g., a sample from a subject, one or more sample compartments, an instructional material which describes performance of a method of the invention and specific controls/standards.

The instructions can be, for example, printed instructions for performing the assay for evaluating the results.

The means for isolating a biological sample from a subject can comprise one or more reagents that can be used to obtain a fluid or tissue from a subject. The means for obtaining a biological sample from a subject may also comprise means for isolating peripheral blood mononuclear cells from a blood sample, for example by positive selection of the monocytes or by negative selection in which all other cell types other than monocytes are removed.

The kits of the invention may further a TNFα inhibitor.

Preferably, the kit is designed for use with a human subject.

Kits of the invention can be used to determine if a subject with RA will be effectively responsive to a TNFa inhibitor. These kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a protein or a biomarker (HLA-DRB1 SE, IL-4R I50V SNP, and/or FcγRIIb I232 SNP) gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, e.g., avidin or streptavidin, bound to a reporter molecule, such as an enzymatic, florescent, or radioisotope label.

Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the invention have a number of embodiments. A typical embodiment is a kit comprising a container, a label on the container, and a composition contained within the container, wherein the composition includes one or more polynucleotides that hybridize to a complement of the IL-4R I50V SNP, and/or FcγRIIb I232 SNP and/or of HLA-DRB1 SE under stringent conditions, and the label on the container indicates that the composition can be used to evaluate the presence of IL-4R I50V SNP, and/or FcγRIIb I232 SNP, and/or of HLA-DRB1 SE in a sample, and wherein the kit includes instructions for using the polynucleotide(s) for evaluating the presence of the SNP and/or SE RNA or DNA in a particular sample type.

Another aspect is a kit comprising a container, a label on the container, and a composition contained within the container, wherein the composition includes a primary antibody that binds to a protein or autoantibody biomarker, and the label on the container indicates that the composition can be used to evaluate the presence of such proteins or antibodies in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label, e.g., an enzymatic label.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In further specific embodiments, for antibody-based kits, the kit can comprise, for example: (1) a first antibody (e.g., attached to a solid support) that binds to a biomarker protein; and, optionally, (2) a second, different antibody that binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein-stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container, and all of the various containers can be included within a single package, along with instructions for interpreting the results of the assays performed using the kit.

Also provided by the invention are articles of manufacture containing materials useful for the treatment of the RA. The article of manufacture comprises a container and a label or package insert on or associated with the container. In this aspect, the package insert is on or associated with the container. Suitable containers include, for example, bottles, vials, syringes, etc. The containers may be formed from a variety of materials such as glass or plastic. The container holds or contains the antagonist that is effective for treating the RA and may have a sterile access port (for example, the container may be an intravenous solution bag or a vial having a stopper pierceable by a hypodermic injection needle). At least one active agent in the composition is the B-cell antagonist. The label or package insert indicates that the composition is used for treating RA in a subject eligible for treatment with specific guidance regarding dosing amounts and intervals of antagonist and any other medicament being provided.

The kits and articles of manufacture herein also include information, for example in the form of a package insert or label, indicating that the composition is used for treating RA where the genotype(s) showing the polymorphism and/or SE herein are detected in a genetic sample from the patient with the disease. The insert or label may take any form, such as paper or electronic media, for example, a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the antagonist may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, and patent information.

In a specific embodiment of the invention, an article of manufacture is provided comprising, packaged together, a pharmaceutical composition comprising a TNFa inhibitor and a pharmaceutically acceptable carrier and a label stating that the inhibitor or pharmaceutical composition is indicated for treating patients with RA from which a genetic sample has been obtained showing the presence of a IL-4R I50V SNP, and/or FcγRIIb I232 SNP and/or HLA-DRB1 SE allele. This can be shown by assessing genetic expression as a biomarker of a IL-4R I50V SNP, and/or FcγRIIb I232 SNP and/or HLA-DRB1 SE allele.

Also the invention provides a method for manufacturing a TNFα inhibitor or a pharmaceutical composition thereof comprising combining in a package the TNFα inhibitor or pharmaceutical composition and a label stating that the TNFα inhibitor or pharmaceutical composition is indicated for treating patients with RA from which a genetic sample has been obtained showing the presence of an HLA-DRB1 SE, an FcγRIIb I232 SNP, and/or an IL-4R I50V SNP. Alternatively, specific alleles associated with each SNP correlated with a response may be individually recited. The label may further state that this can be shown by assessing genetic expression as a biomarker of a IL-4R I50V SNP, and/or FcγRIIb I232 SNP, and/or HLA-DRB1 SE. Notably, each of the genetic markers identified in the invention may be described individually or in combination with one another.

The contents of all references, patents and published patent applications cited throughout this application are incorporated herein by reference

This invention is further illustrated by the following example, which should not be construed as limiting.

### EXAMPLES

### Example 1

### Response of Early Rheumatoid Arthritis to Treatment with Adalimumab Plus Methotrexate Vs. Methotrexate Alone: Predicting Clinical Response by Genetic Marker Analysis

The following study and examples 2-4, examined the contribution of genetic factors to the treatment of rheumatoid arthritis (RA) with a TNFα inhibitor, adalimumab, plus methotrexate versus methotrexate alone.

The objective of the study was to prospectively analyze the association of 3 genetic risk factors (HLA-DRB1 shared epitope (SE), FcγRIIb, and IL-4R) for severe RA with clinical disease activity after 26 weeks of combination therapy with adalimumab (ADA) and methotrexate (MTX) or MTX monotherapy in a substudy of OPTIMA (A Multicentre, Randomized, Double Period, Double-Blind Study to Determine the Optimal Protocol for Treatment Initiation With Methotrexate and Adalimumab Combination Therapy in Patients With Early Rheumatoid Arthritis).

OPTIMA is an ongoing 78-week study with 26- and 52-week periods. Eligible patients had RA<1 year (1987-revised ACR classification), 28-joint Disease Activity Score (DAS28)>3.2, ≧6 swollen joints (TJC68≧6), and ≧8 tender joints (SSJC66≧8). Patients had elevated erythrocyte sedimentation rate (ESR)≧28 mm/h or C-reactive protein (CRP)≧1.5 mg/dL and ≧1 of the following: >1 erosion, rheumatoid-factor positive (RF+), or anti-cyclic citrullinated peptide antibody positive (anti-CCP+). Exclusion criteria included prior exposure to systemic anti-TNF therapies, treatment with MTX or >2 disease-modifying anti-rheumatic drugs (DMARDs), other acute inflammatory joint diseases, or steroidal or surgical treatment within 4 weeks or 2 months, respectively.

Patients were genotyped by allele-specific polymerase chain reaction (PCR) and direct sequencing as needed for the presence of the HLA-DRB1 SE (homo- or heterozygosity), the FcγRIIb I232T single nucleotide polymorphism (SNP) (via allele-specific PCR using Assay-on-Demand (Applied Biosystems)), and the IL-4R I50V SNP (via allele-specific PCR using Assay-on-Demand (Applied Biosystems)).

As shown in FIG. 1, MTX-naïve patients were randomized initially randomized 1:1 to receive oral MTX (escalated to 20 mg) weekly, plus adalimumab (ADA) 40 mg every other week or placebo (PBO) by subcutaneous injection, for the first 26 weeks of treatment (Period 1). In Period 2, those combination therapy responders, e.g., meeting DAS28 low disease activity (LDA) criteria (DAS28<3.2), were re-randomized 1:1 to remain on ADA+MTX or to “step down” to PBO+MTX for Weeks 26-78. In the initial PBO+MTX monotherapy group, subjects who achieved DAS28 LDA after Period 1 remained blinded on PBO+MTX for Period 2. Any subject who failed to meet the DAS28 LDA criteria at Weeks 22 and/or 26 received open-label ADA+MTX beyond week 26. Genetic data were associated with Week-26 clinical response.

For clinical assessment, the percentage of subjects achieving a 20%, 50%, and 70% improvement from baseline ACR scores was determined after Week 26 using a non-responder imputation approach. DAS28(CRP)¹ scores were evaluated at Week 26. The proportion of subjects achieving LDA (DAS28<3.2) and remission criteria (DAS28<2.6) was determined using a non-responder imputation approach.

For statistical analyses, Allele distribution between treatment groups was evaluated using the chi-square test, or Fisher's exact test in cases where data were sparse. The chi-square test was used to compare the proportion of subject achieving ACR20/50/70 and DAS28 LDA (<3.2) or remission (<2.6) within and between treatment groups.

The study population included 1032 patients randomized to PBO+MTX (N=517) or ADA+MTX (N=515) for the first 26 weeks. During Period 1, 106 subjects (10%) discontinued prematurely (PBO+MTX: N=57, 11%; and ADA+MTX: N=49, 10%). Of the 1032 subjects enrolled, 894 subjects (87%) had genetic data available for this subanalysis (PBO+MTX: N=451, 87%; and ADA+MTX: N=443, 86%).

As seen below in Table 1, all 3 genetic factors were in Hardy Weinberg equilibrium in both treatment groups. The MTX and ADA+MTX groups did not differ significantly in the percentages of patients carrying the HLA-DRB1 SE (63% vs. 67%, respectively) or in the percentages of patients carrying 0, 1, or 2 copies of the SE (MTX: 37%, 48%, 15%; ADA+MTX: 33%, 49%, 19%). Similarly, the percentages of patients with IL-4R I50V alleles did not differ significantly between the MTX and ADA+MTX groups (A allele homozygosity: 29% vs. 33%; G allele homozygosity: 20% vs. 20%; heterozygosity: 52% vs. 47%). In contrast, and by chance, distribution of the FcγRIIb I232T allele significantly differed between groups, excluding this allele from further analysis. Analysis of the FcγRIIb I232T allele is provided in Example 4.

TABLE 1
Allele Distribution

| | PBO + MTX | ADA + MTX | Total | P |
|---|---|---|---|---|
| Genotype | N = 451 | N = 443 | N = 894 | value^{a} |
| HLA-DRB1 SE | | | | 0.28 |
| (copy #) | | | | |
| 0 | 167 (37.0%) | 145 (32.7%) | 312 (34.9%) | |
| 1 | 215 (47.7%) | 216 (48.8%) | 431 (48.2%) | |
| 2 | 69 (15.3%) | 82 (18.5%) | 151 (16.9%) | |
| IL-4R | | | | 0.38 |
| AA (I50I) | 130 (28.8%) | 145 (32.7%) | 275 (30.8%) | |
| AG (I50V) | 233 (51.7%) | 210 (47.4%) | 443 (49.6%) | |
| GG (V50V) | 88 (19.5%) | 88 (19.9%) | 176 (19.7%) | |
| FcγRIIb | | | | 0.03 |
| TT (I232I) | 333 (73.8%) | 360 (81.3%) | 693 (77.5%) | |
| TC (I232T) | 111 (24.6%) | 77 (17.4%) | 188 (21.0%) | |
| CC (T232T) | 7 (1.6%) | 6 (1.4%) | 13 (1.5%) | |

^{a}P values based on chi-square test.

The baseline demographics and disease characteristics of subjects stratified by HLA-DRB1 SE and IL-4R alleles are presented in Tables 2 and 3, respectively.

TABLE 2
Baseline Demographics and Disease Characteristics
by HLA-DRB1 SE Copy Number

| | 0 copies SE | | 1 copy SE | | 2 copies SE | |
|---|---|---|---|---|---|---|
| | N = 312 | | N = 431 | | N = 151 | |
| Sex, n/N (%) | 245/312 | (79%) | 303/431 | (70%) | 106/151 | (70%) |
| female | | | | | | |
| Race, n/N (%) | 257/312 | (82%) | 398/431 | (92%) | 146/151 | (97%) |
| white | | | | | | |
| Age, mean (SD) | 51.0 | (13.1) | 51.4 | (13.9) | 47.6 | (14.6) |
| years | | | | | | |
| Smoker, nN (%) | 150/312 | (48%) | 232/431 | (54%) | 76/151 | (50%) |
| RF+, n/N (%) | 260/310 | (83%) | 375/425 | (87%) | 138/150 | (91%) |
| RF >50 IU, | 177/310 | (57%) | 299/425 | (70%) | 109/150 | (73%) |
| n/N (%) | | | | | | |
| anti-CCP+, | 227/311 | (73%) | 370/426 | (87%)* | 139/149 | (93%) |
| n/N (%) | | | | | | |
| RF+ and anti- | 216/309 | (70%) | 347/422 | (82%) | 133/149 | (89%) |
| CCP+, n/N (%) | | | | | | |
| CRP, mean (SD) | 2.71 | (3.19) | 2.99 | (3.25) | 3.04 | (3.05) |
| mg/dl | | | | | | |
| DAS28, | 6.1 | (0.99)^{a} | 6.0 | (0.99)^{b} | 6.0 | (0.86)^{c} |
| mean (SD) | | | | | | |
| HAQ, mean (SD) | 1.6 | (0.69)^{d} | 1.6 | (0.67)^{e} | 1.7 | (0.60) |

*Significant difference between treatment groups (193/213, 91% PBO + MTX, 177/213, 83% ADA + MTX, P = 0.02).
^{a}N = 301;
^{b}N = 428;
^{c}N = 149;
^{d}N = 311;
^{e}N = 430.

TABLE 3
Baseline Demographics and Disease Characteristics by IL-4R Alleles

| | AA | | AG | | GG | |
|---|---|---|---|---|---|---|
| | N = 275 | | N = 443 | | N = 176 | |
| Sex, n/N (%) | 201/275 | (73%) | 320/443 | (72%) | 133/176 | (76%) |
| female | | | | | | |
| Race, n/N (%) | 250/275 | (91%) | 395/443 | (89%) | 156/176 | (89%) |
| white | | | | | | |
| Age, mean (SD) | 50.6 | (13.2) | 50.8 | (14.0) | | |
| years | | | | | | |
| Smoker, n/N (%) | 139/275 | (51%) | 227/443 | (51%) | 92/176 | (52%) |
| RF+, n/N (%) | 241/271 | (89%) | 385/441 | (87%) | 147/173 | (85%) |
| RF >50 IU, | 177/271 | (65%) | 297/441 | (67%) | 111/173 | (64%) |
| n/N (%) | | | | | | |
| anti-CCP+, | 226/271 | (83%) | 368/441 | (83%) | 142/174 | (82%) |
| n/N (%) | | | | | | |
| RF+ and anti- | 214/268 | (80%) | 348/440 | (79%) | 134/172 | (78%) |
| CCP+, n/N (%) | | | | | | |
| CRP, mean (SD) | 2.82 | (3.03) | 3.00 | (3.34) | 2.80 | (3.09) |
| mg/dl | | | | | | |
| DAS28, | 6.0 | (0.99)^{a} | 6.1 | (0.94)^{b} | 6.0 | (1.00)^{c} |
| mean (SD) | | | | | | |
| HAQ, mean (SD) | 1.6 | (0.65)^{d} | 1.6 | (0.68)^{e} | 1.6 | (0.65) |

^{a}N = 267;
^{b}N = 436;
^{c}N = 175;
^{d}N = 274;
^{e}N = 442.

Overall, subjects receiving ADA+MTX combination therapy responded significantly better to 26 weeks of treatment compared with subjects in the PBO+MTX treatment group.

As shown below in FIG. 2 and Tables 4a-4c, the number of HLA-DRB1 SE copies was associated with clinical response. However, whereas increased copy numbers were associated with decreased achievement of American College of Rheumatology rating scale improvements (ACR20, ACR50, and ACR70) and 28-joint Disease Activity Score remission criteria for the MTX group, increased copy numbers were significantly and directly correlated with better clinical response for the ADA+MTX group (e.g., ACR50 for 0, 1, and 2 copies: 40%, 33%, and 29% for the MTX group vs. 42%, 53%, and 65% for the ADA+MTX group). These data show that in subjects with at least 1 copy of the SE, combination therapy with ADA+MTX was associated with significantly improved ACR20/50/70 response rates compared with MTX monotherapy and that cumulative increases in the ACR responses to ADA+MTX were observed in subjects with 1 or 2 copies of the SE allele.

TABLE 4a
ACR20 Response Rates at Week 26, by Presence of HLA-DRB1 SE

| n/N, (%) | ACR20 | | | |
|---|---|---|---|---|
| subjects | 0 copies | 1 copy | 2 copies | P value^{#} |
| ADA + MTX | 89/145 | 153/216 | 67/82 | 0.005 |
| | (61%) | (71%) | (82%) | |
| PBO + MTX | 105/167 | 115/215 | 36/69 | 0.13 |
| | (63%) | (54%) | (52%) | |
| P value* | 0.79 | <0.001 | <0.001 | |

^{#}P value comparing within treatment group responses;
*P value for differences between treatment groups; P values based on chi-square test.

TABLE 4b
ACR50 Response Rates at Week 26, by Presence of HLA-DRB1 SE

| n/N, (%) | ACR50 | | | |
|---|---|---|---|---|
| subjects | 0 copies | 1 copy | 2 copies | P value^{#} |
| ADA + MTX | 61/145 | 114/216 | 53/82 | 0.004 |
| | (42%) | (53%) | (65%) | |
| PBO + MTX | 66/167 | 71/215 | 20/69 | 0.23 |
| | (40%) | (33%) | (29%) | |
| P value* | 0.65 | <0.001 | <0.001 | |

^{#}P value comparing within treatment group responses;
*P value for differences between treatment groups; P values based on chi-square test.

TABLE 4c
ACR70 Response Rates at Week 26, by Presence of HLA-DRB1 SE

| n/N, (%) | ACR70 | | | |
|---|---|---|---|---|
| subjects | 0 copies | 1 copy | 2 copies | P value^{#} |
| ADA + MTX | 38/145 | 75/216 | 40/82 | 0.003 |
| | (26%) | (35%) | (49%) | |
| PBO + MTX | 31/157 | 36/215 | 9/69 | 0.59 |
| | (19%) | (17%) | (13%) | |
| P value* | 0.11 | <0.001 | <0.001 | |

^{#}P value comparing within treatment group responses;
*P value for differences between treatment groups; P values based on chi-square test.

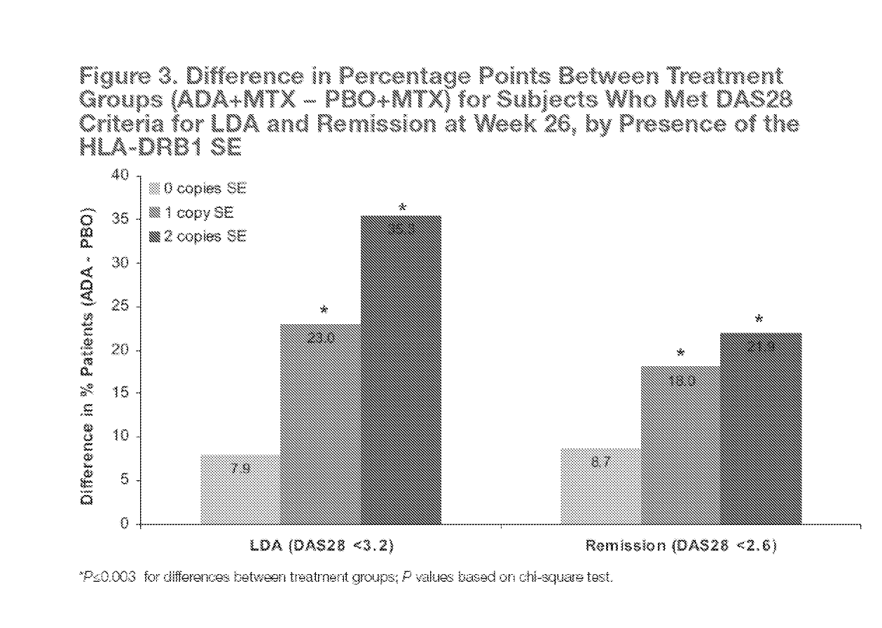

Furthermore, as shown in FIG. 3 and Table 5 below, in subjects with at least 1 copy of the SE, combination therapy with ADA+MTX was associated with significantly improved DAS28 responses compared with MTX monotherapy. Cumulative increases in the proportion of ADA+MTX subjects meeting DAS28 LDA criteria were observed in subjects with 1 or 2 copies of the SE allele. Presence of the SE was not associated with DAS28 responses to MTX monotherapy.

TABLE 5
Proportion of Subjects Meeting DAS28 Criteria for LDA
and Remission at Week 26, by Presence of HLA-DRB1 SE

| | LDA | | | | Remission | | | |
|---|---|---|---|---|---|---|---|---|
| n/N (%) | DAS28 <3.2 | | | | DAS28 <2.6 | | | |
| subjects | 0 copies | 1 copy | 2 copies | P value^{#} | 0 copies | 1 copy | 2 copies | P value^{#} |
| ADA + MTX | 54/145 | 100/216 | 48/82 | 0.008 | 37/145 | 74/216 | 31/82 | 0.10 |
| | (37%) | (46%) | (59%) | | (26%) | (34%) | (38%) | |
| PBO + MTX | 49/167 | 50/215 | 16/69 | 0.36 | 28/167 | 35/215 | 11/69 | 0.99 |
| | (30%) | (23%) | (24%) | | (17%) | (16%) | (16%) | |
| P value* | 0.16 | <0.001 | <0.001 | | 0.06 | <0.001 | 0.003 | |

^{#}P value comparing within treatment group responses;
*P value for differences between treatment groups;
P values based on chi-square test.

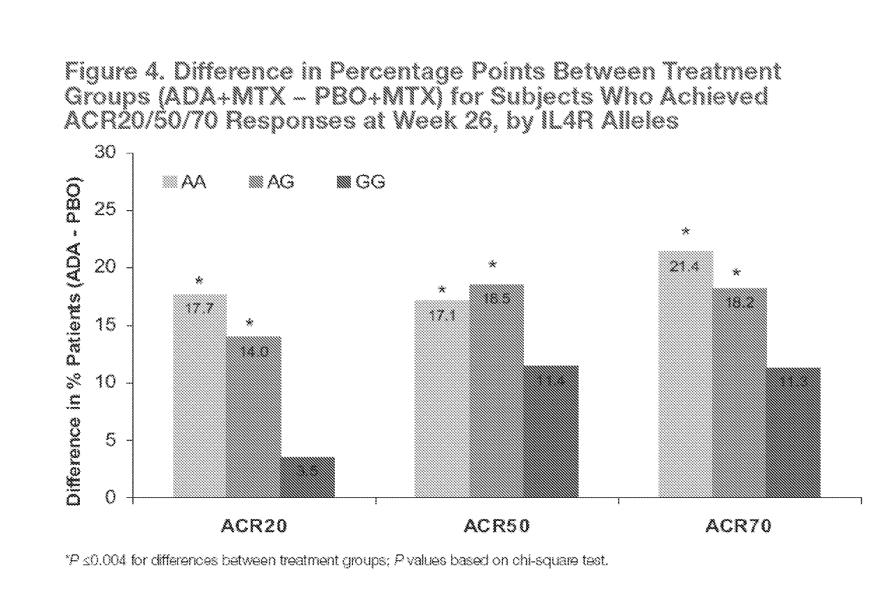

As seen below in FIG. 4 and Tables 6a-6c, a significantly enhanced clinical response was observed for patients on ADA+MTX who were either homozygous or heterozygous for the IL-4R I50 alleles but not in patients with two IL-4R V50 alleles. Subjects bearing at least 1 IL-4R I50 allele (AA or AG) demonstrated significantly improved ACR responses to combination therapy with ADA+MTX relative to MTX monotherapy. Subjects treated with ADA+MTX who were either homozygous (AA) or heterozygous (AG) for the IL-4R I50 allele had a significantly enhanced ACR20 response compared with ADA+MTX subjects with the IL-4R V50V allele (GG). The IL-4R alleles were not associated with a differential response to PBO+MTX as assessed by ACR20/50/70 response rates.

TABLE 6a
ACR20 Response Rates at Week 26, by IL-4R Alles

| n/N, (%) | ACR70 | | | |
|---|---|---|---|---|
| Subjects | AA | AG | GG | P value^{#} |
| ADA + MTX | 50/145 | 77/210 | 26/88 | 0.50 |
| | (35%) | (37%) | (30%) | |
| PBO + MTX | 17/130 | 42/233 | 16/88 | 0.39 |
| | (13%) | (19%) | (18%) | |
| P value* | <0.001 | <0.001 | 0.08 | |

TABLE 6b
ACR50 Response Rates at Week 26, by IL-4R Alles

| n/N, (%) | ACR50 | | | |
|---|---|---|---|---|
| Subjects | AA | AG | GG | P value^{#} |
| ADA + MTX | 75/145 | 110/210 | 43/88 | 0.86 |
| | (52%) | (52%) | (49%) | |
| PBO + MTX | 45/130 | 79/233 | 33/88 | 0.83 |
| | (35%) | (34%) | (38%) | |
| P value* | 0.004 | <0.001 | 0.13 | |

TABLE 6c
ACR70 Response Rates at Week 26, by IL-4R Alles

| n/N, (%) | ACR70 | | | |
|---|---|---|---|---|
| Subjects | AA | AG | GG | P value^{#} |
| ADA + MTX | 50/145 | 77/210 | 26/88 | 0.50 |
| | (35%) | (37%) | (30%) | |
| PBO + MTX | 17/130 | 42/233 | 16/88 | 0.39 |
| | (13%) | (19%) | (18%) | |
| P value* | <0.001 | <0.001 | 0.08 | |

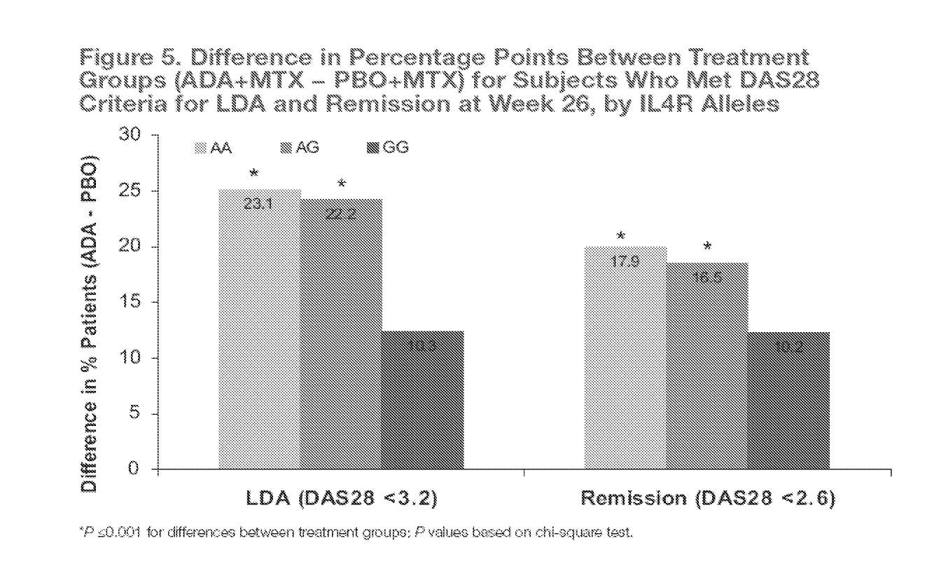

As seen below in FIG. 5 and Table 7, for ACR responses, subjects bearing at least 1 IL-4R I50 allele (AA or AG) demonstrated significantly improved DAS28 responses to combination therapy with ADA+MTX relative to MTX monotherapy. A higher proportion of subjects treated with ADA+MTX who were either homozygous (AA) or heterozygous (AG) for the I50 allele achieved a DAS28 LDA compared with ADA+MTX subjects with the IL-4R V50V allele (GG). Conversely, presence of the I50 allele was associated with a trend towards a decreased proportion of subjects treated with MTX monotherapy who met DAS28 LDA and remission criteria.

TABLE 7
Proportion of Subjects Meeting DAS28 Criteria for
LDA and Remission at Week 26, byIL-4R Alleles

| | LDA | | | | Remission | | | |
|---|---|---|---|---|---|---|---|---|
| n/N (%) | DAS28 <3.2 | | | | DAS28 <2.6 | | | |
| subjects | AA | AG | GG | P value^{#} | AA | AG | GG | P value^{#} |
| ADA + MTX | 68/145 | 99/210 | 35/88 | 0.47 | 46/145 | 68/210 | 28/88 | 0.99 |
| | (47%) | (47%) | (40%) | | (32%) | (32%) | (32%) | |
| PBO + MTX | 31/130 | 58/233 | 26/88 | | 18/130 | 37/233 | 19/88 | 0.30 |
| | (24%) | (25%) | (30%) | 0.61 | (14%) | (16%) | (22%) | |
| P value* | <0.001 | <0.001 | 0.15 | | <0.001 | <0.001 | 0.13 | |

^{#}P value comparing within treatment group responses;
*P value for differences between treatment groups;
P values based on chi-square test.

In conclusion, the HLA-DRB1 shared epitope and IL-4R I50V polymorphism were independently associated with differential treatment responses in patients with early RA. The presence of the HLA-DRB1 shared epitope or IL-4R I50 allele increased clinical responses in patients treated with adalimumab plus methotrexate. The HLA-DRB1 shared epitope and the IL-4R I50 allele were independently associated with enhanced clinical responses following 26 weeks of treatment with adalimumab plus methotrexate compared with methotrexate monotherapy. Hence the results of the study show that the HLA-DRB1 SE and the IL-4R I50V contributed to the clinical response to ADA+MTX therapy. Thus genetic marker analysis can facilitate personalized medicine in patients with early RA, and may be used to predict whether or not a subject will be responsive to treatment of RA with a TNFα inhibitor.

### Example 2

### Impact of Genetic Interactions on Response to Adalimumab Plus Methotrexate Versus Methotrexate Alone: Six Months Results of the OPTIMA Trial

Identification of genetic factors that affect rheumatoid arthritis (RA) disease severity and response to treatment can guide personalized therapeutic approaches. To explore the impact of candidate genetic factors on changes in disease activity, the following study examined the contribution of genetic factors to the treatment of rheumatoid arthritis (RA) with adalimumab plus methotrexate versus methotrexate alone.

OPTIMA is an ongoing 78-week study with 26- and 52-week periods. Details of the study design and patient eligibility/exclusion criteria are described above in Example 1. Briefly, eligible patients had RA<1 year, DAS28>3.2, ≧6 SJC, ≧8 TJC. ESR≧28 mm/h or CRP≧1.5 mg/dL, and ≧1 of the following: >1 erosion, RF+, or anti-CCP+ (see above). MTX-naïve patients were randomized to ADA 40 mg every other week+MTX or placebo (PBO)+MTX (see above). Patients were genotyped by allele-specific polymerase chain reaction (PCR) and direct sequencing as needed for the presence of the HLA-DRB1 shared epitope (SE), the FcγRIIb I232T single nucleotide polymorphism (SNP), and the IL-4R I50V SNP. Clinical responses to 26 weeks of treatment were examined by genetic background for each allele independently, and in the allele combinations for SE and IL-4R.

Subjects in the treatment groups demonstrated a comparable distribution of 0, 1, or 2 copies of the HLA-DRB1 SE (PBO+MTX: 37%, 48%, 15%; ADA+MTX: 33%, 49%, 19%, respectively, P=0.28, see Table 1 above). Likewise, the IL-4R alleles, AA, AG, and GG, were distributed similarly between treatment groups (PBO+MTX: 29%, 52%, 20%; ADA+MTX: 33%, 47%, 20%, respectively, P=0.38, see Table 1 above). The FcγRIIb alleles, however, were dissimilarly appropriated between treatment groups (see Table 1 above), and no further analysis was conducted on this SNP in this example but are provided in Example 4.

Presence of the SE did not affect treatment response to MTX alone (e.g., ACR50 of 40%, 33%, and 29% for 0, 1, or 2 copies, P=0.23, see Table 4 above). Conversely, treatment response rates were correspondingly enhanced with increasing copies of the SE in subjects receiving ADA+MTX (ACR50 of 42%, 53%, and 65% for 0, 1, 2 SE, P=0.004, see Table 4 above). Thus, presence of 1 copy of the SE afforded a 20% increase in ACR50 for ADA+MTX subjects relative to the PBO+MTX group (P<0.001), and 2 copies of SE increased ACR50 in ADA+MTX by 36% over PBO+MTX (P<0.001). See Table 4 above.

Similarly, clinical responses to MTX were not affected by IL-4R alleles, while treatment outcomes with ADA+MTX was enhanced in subjects with AA or AG IL-4R alleles. See Table 6 above.

Examination of treatment responses for the SE and IL-4R allele combinations in the PBO+MTX group shows no alteration in responses by genotype, supporting results from analysis of the individual alleles. However, in the absence of the SE, IL-4R genotype affects treatment response to ADA+MTX, while presence of the SE masks effects of the IL-4R alleles (See Table 8).

TABLE 8
Genetic Interaction between HLA-DRB1 SE and IL-4R

| ACR50, | IL-4R | HLA-DRB1 SE copy # | | | | | |
|---|---|---|---|---|---|---|---|
| n/N (%) | allele | 0 | | 1 | | 2 | |
| PBO + MTX | AA | 15/34 | (44%) | 23/56 | (41%) | 7/23 | (30%) |
| | AG | 33/78 | (42%) | 34/92 | (37%) | 12/32 | (38%) |
| | GG | 18/38 | (47%) | 14/35 | (40%) | 1/2 | (50%) |
| ADA + MTX | AA | 27/46 | (59%) | 32/57 | (56%) | 16/23 | (70%) |
| | AG | 24/55 | (44%) | 58/94 | (62%) | 28/40 | (70%) |
| | GG | 10/26 | (39%) | 24/41 | (59%) | 9/12 | (75%) |

Results reported herein show that clinical responses to adalimumab plus methotrexate are independently affected by both the HLA-DRB1 shared epitope and IL-4R alleles, while there was no impact of genotype on the response to methotrexate monotherapy. In addition, there is an interaction between the HLA-DRB1 shared epitope and IL-4R alleles in response to treatment with adalimumab plus methotrexate.

### Example 3

### Impact of Genetic Interactions on Response to Adalimumab Plus Methotrexate Versus Methotrexate Alone: Six Months Results of the OPTIMA Trial

### Background

Identification of genetic factors that affect rheumatoid arthritis (RA) disease severity and response to treatment can guide personalized therapeutic approaches. While specific genetic factors have been implicated in the susceptibility to and severity of rheumatoid arthritis (RA), the effect of genetic components on response to biologic RA treatments has not been widely explored.

### Objective

The objective of this study was to explore the impact of candidate genetic factors on changes in disease activity following treatment with adalimumab (ADA) plus methotrexate (MTX) or MTX alone. In addition, the impact of candidate genetic factors on changes in disease activity in patients with early RA following treatment with adalimumab (ADA) plus methotrexate (MTX) or MTX alone was also explored.

### Methods

### Study Design (FIG. 1)

OPTIMA was a Phase 4 multicentre, 2-period, doubleblind, placebo-controlled randomized clinical trial to determine the Optimal Protocol for Treatment Initiation with Methotrexate and Adalimumab combination therapy in patients with early RA. Key inclusion criteria for eligible patients were:

1) 18 years of age

2) RA (1987 ACR-classification criteria)<1 year from diagnosis

3) DAS28>3.2

4) TJC68≧8 and SJC66≧6

5) ESR≧28 mm/h or CRP≧1.5 mg/dL

Subjects in this genetic substudy gave additional voluntary written informed consent to participate.

MTX-naïve patients were randomized 1:1 to ADA (40 mg eow)+MTX (titrated to 20 mg/wk by Week 8) or placebo (PBO)+MTX for the first 26 weeks.

Any subject failing to meet LDA (DAS28<3.2) at Week 22 and/or 26 was offered the option to continue treatment with open-label ADA+MTX.

Responder subjects initially treated with ADA+MTX who achieved LDA at Weeks 22 and 26 were re-randomized to compare continued combination therapy vs. ADA withdrawal through Week 78. PBO+MTX subjects with LDA at Weeks 22 and 26 remained blinded on MTX monotherapy.

In summary, OPTIMA is an ongoing 78-week study with 26- and 52-week periods. Eligible patients had RA<1 year, DAS28>3.2, ≧6 SJC, ≧8 TJC. ESR≧28 mm/h or CRP≧1.5 mg/dL, and ≧1 of the following: >1 erosion, RF+, or anti-CCP+. MTX-naïve patients were randomized to ADA 40 mg every other week+MTX or placebo (PBO)+MTX. Patients were genotyped by allele-specific polymerase chain reaction (PCR) and direct sequencing as needed for the presence of the HLA-DRB1 shared epitope (SE), the FcγRIIb I232T single nucleotide polymorphism (SNP), and the IL-4R I50V SNP. Clinical responses to 26 weeks of treatment were examined by genetic background for each allele independently, and in the allele combinations for SE and IL-4R.

### Genetic Analyses

To determine HLA-DRB1 SE homozygosity or heterozygosity, HLA-DRB1 typing was performed in a two step procedure. Firstly, all patients were typed on a low resolution level using the LABType SSO assay (One Lambda Inc.). DRB1*01, *04, *10 and *14 positive patients were subsequently typed on a high resolution level using sequence based typing (AlleleSEQR, Abbott Molecular Diagnostics). In the case of ambiguities, the DRB high-resolution SSO kit from Biotest was additionally used.

In certain instances, high-resolution typing with Protrans S4 Sequencing Kits (Medipro) was used to determine whether a patient has HLA-DRB1 SE homozygosity or heterozygosity.

Allele-specific PCR using Assay-on-Demand (Applied Biosystems) was used to determine IL-4R (A to G [I50V]) SNP.

Allele-specific PCR using Assay-by-Design (Applied Biosystems) was used to determine FcγRIIb (T to C [I232T]) variant.

### Clinical Assessments

The percentage of subjects achieving a 50% improvement in the ACR score from baseline was determined at Week 26 using a non-responder imputation approach. The percentage of subjects achieving DAS28(CRP) remission (DAS28<2.6) was determined at Week 26 using a non-responder imputation approach.

### Statistical Analyses

Allele distribution between treatment groups was evaluated using the chi-square test, or Fisher's exact test in cases where data were sparse. Multivariate logistic regression was used to evaluate the effect of treatment, individual alleles, the interaction between treatment and genetic components, and baseline demographics and disease characteristics on clinical responses at 26 weeks.

### Results

### Study Population

### Subject Disposition

The OPTIMA trial randomized 1032 patients: PBO+MTX: N=517 and ADA+MTX: N=515

During the first 26-week Period, 106 subjects (10%) discontinued prematurely: PBO+MTX: N=57, 11% and ADA+MTX: N=49, 10%

In this genetic substudy, 894 of 1032 subjects (87%) had genotypic data available for this analysis:

PBO+MTX: N=451, 87%

ADA+MTX: N=443, 86%

### Baseline Characteristics

### Randomization was not Stratified Based on the Allele Type:

Subjects in each treatment group showed a similar distribution of the HLA-DRB1 SE and IL-4R allele variants; however the FcγRIIb SNP was unequally distributed and was excluded from further analysis (see Table 9 below), but were analyzed in a separate analysis described in Example 4.

TABLE 9
Allele Distribution.
The Number (%) of Subjects
Expressing the Indicated Genotypes

| | PBO + MTX | | ADA + MTX | | Total | | P |
|---|---|---|---|---|---|---|---|
| Genotype | N = 451 | | N = 443 | | N = 894 | | value^{a} |
| HLA-DRB1 SE | | | | | | | 0.28 |
| (copy #) | | | | | | | |
| 0 | 167 | (37%) | 145 | (33%) | 312 (35%) | | |
| 1 | 215 | (48%) | 216 | (49%) | 431 (48%) | | |
| 2 | 69 | (15%) | 82 | (19%) | 151 (17%) | | |
| IL-4R | | | | | | | 0.38 |
| AA (I50/I50) | 130 | (29%) | 145 | (33%) | 275 (31%) | | |
| AG (I50/I50V) | 233 | (52%) | 210 | (47%) | 443 (50%) | | |
| GG (I50V/I50V) | 88 | (20%) | 88 | (20%) | 176 (20%) | | |
| FcγRIIb | | | | | | | 0.03 |
| TT (I232/I232) | 333 | (74%) | 360 | (81%) | 693 | (78%) | |
| TC (I232/I232T) | 111 | (25%) | 77 | (17%) | 188 | (21%) | |
| CC (I232T/I232T) | 7 | (1.6%) | 6 | (1.4%) | 13 | (1.5%) | |

^{a}P values based on chi-square test.

Baseline demographics and disease characteristics were similar among allele variants across treatment groups (Tables 10 and 11):

An increasing percentage of anti-CCP+ patients was noted with increasing copies of the SE.

More smokers were identified in the PBO+MTX group for patients with 1 copy of the SE compared with ADA+MTX patients with 1 SE allele.

TABLE 10
Baseline Demographics and Disease Characteristics
by SE Copy Number

| | 0 copies SE | | 1 copy SE | | 2 copies SE | |
|---|---|---|---|---|---|---|
| | N = 312 | | N = 431 | | N = 151 | |
| Sex, n/N (%) | 245/312 | (79%) | 303/431 | (70%) | 106/151 | (70%) |
| female | | | | | | |
| Race, n/N (%) | 257/312 | (82%) | 398/431 | (92%) | 146/151 | (97%) |
| white | | | | | | |
| Age, mean (SD) | 51.0 | (13.1) | 51.4 | (13.9) | 47.6 | (14.6) |
| years | | | | | | |
| Smoker, n/N (%) | 150/312 | (48%) | 232/431 | (54%) | 76/151 | (50%) |
| RF+, n/N(%) | 260/310 | (83%) | 375/425 | (87%) | 138/150 | (91%) |
| RF >50 IU, | 177/310 | (57%) | 299/425 | (70%) | 109/150 | (73%) |
| n/N (%) | | | | | | |
| anti-CCP+, | 227/311 | (73%) | 370/426 | (87%)* | 139/149 | (93%) |
| n/N (%) | | | | | | |
| RF+ and anti- | 216/309 | (70%) | 347/422 | (82%) | 133/149 | (89%) |
| CCP+, | | | | | | |
| n/N (%) | | | | | | |
| CRP, mean (SD) | 2.71 | (3.19) | 2.99 | (3.25) | 3.04 | (3.05) |
| mg/dl | | | | | | |
| DAS28, | 6.1 | (0.99)^{a} | 6.0 | (0.99)^{b} | 6.0 | (0.86)^{c} |
| mean (SD) | | | | | | |
| HAQ, mean (SD) | 1.6 | (0.69)^{d} | 1.6 | (0.67)^{e} | 1.7 | (0.60) |

*Significant difference between treatment groups (193/213, 91% PBO + MTX, 177/213, 83% ADA + MTX, P = 0.02).
^{a}N = 301;
^{b}N = 428;
^{c}N = 149;
^{d}N = 311;
^{e}N = 430.

TABLE 11
Baseline Demographics and Disease Characteristics by IL-4R Alleles

| | AA | | AG | | GG | |
|---|---|---|---|---|---|---|
| | N = 275 | | N = 443 | | N = 176 | |
| Sex, n/N (%) | 201/275 | (73%) | 320/443 | (72%) | 133/176 | (76%) |
| female | | | | | | |
| Race, n/N (%) | 250/275 | (91%) | 395/443 | (89%) | 156/176 | (89%) |
| white | | | | | | |
| Age, mean (SD) | 50.6 | (13.2) | 50.8 | (14.0) | 50.3 | (14.2) |
| years | | | | | | |
| Smoker, n/N (%) | 139/275 | (51%) | 227/443 | (51%) | 92/176 | (52%) |
| RF+, n/N (%) | 241/271 | (89%) | 385/441 | (87%) | 147/173 | (85%) |
| RF >50 IU, | 177/271 | (65%) | 297/441 | (67%) | 111/173 | (64%) |
| n/N (%) | | | | | | |
| anti-CCP+, | 226/271 | (83%) | 368/441 | (83%) | 142/174 | (82%) |
| n/N (%) | | | | | | |
| RF+ and anti- | 214/268 | (80%) | 348/440 | (79%) | 134/172 | (78%) |
| CCP+, | | | | | | |
| n/N (%) | | | | | | |
| CRP, mean (SD) | 2.82 | (3.03) | 3.00 | (3.34) | 2.80 | (3.09) |
| mg/dl | | | | | | |
| DAS28, | 6.0 | (0.99)^{a} | 6.1 | (0.94)^{b} | 6.0 | (1.00)^{c} |
| mean (SD) | | | | | | |
| HAQ, mean (SD) | 1.6 | (0.65)^{d} | 1.6 | (0.68)^{e} | 1.6 | (0.65) |

^{a}N = 267;
^{b}N = 436;
^{c}N = 175;
^{d}N = 274;
^{e}N = 442.

### Treatment Response

To control for possible confounding variables, a multivariate regression analysis was employed to explore the influence of the genetic factors with baseline demographic and disease state variables. In multivariate regression, the treatment effect for ADA+MTX was significant (P<0.001) for achieving both ACR50 and DAS28 remission at Week 26.

### SE Copy Number

### ACR50

An inverse pattern of response rates was observed in the 2 treatment groups: ACR50 responses in the PBO+MTX group showed a decreasing trend with SE multiplicity (Table 4), while ACR50 response rates to ADA+MTX increased in subjects with increasing presence of the SE (Table 4).

Because of the inverse relationship between SE copy number and treatment response in the 2 treatment groups, further multivariate models were conducted within each treatment group. It was discovered that, when accounting for the baseline variables of sex, smoker, RF+, anti-CCP+, TJC68, and DAS28, there was a significant effect of SE copy number within the PBO+MTX group (OR [95% confidence interval, CI]: 0.469 [0.247, 0.893] for 2× vs. 0×SE). In addition, in the ADA+MTX group, it was discovered that the effect of SE copy number was also significant (OR [95% CI]: 2.048 [1.127, 3.722] for 2× vs. 0×SE).

### DAS28 Remission

Among PBO+MTX subjects, there was no effect of SE_copy number on DAS28 remission (Table 5). However, a pattern of increasing DAS28 response rates was observed in ADA+MTX subjects with increasing copies of the SE (Table 5). In addition, within either the PBO+MTX or ADA+MTX groups, there was no significant effect of SE copy number on DAS28 remission by multivariate regression.

### IL-4R Alleles

### ACR50

ACR50 at Week 26 was not meaningfully influenced by IL-4R alleles for subjects receiving either ADA+MTX or PBO+MTX (Table 6).

### DAS28 Remission

IL-4R alleles did not influence the DAS28 remission response rate at Week 26 for subjects within either PBO+MTX or ADA+MTX treatment groups (Table 7).

These observations were supported by multivariate regression, with no significant effect of IL-4R alleles on either treatment response variable.

### Combined Allele Effects

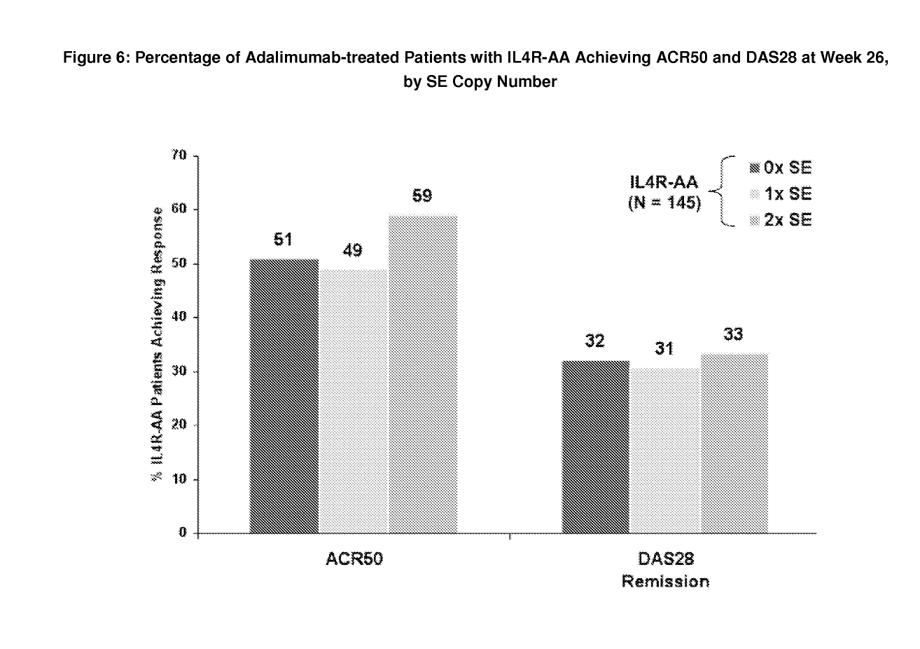

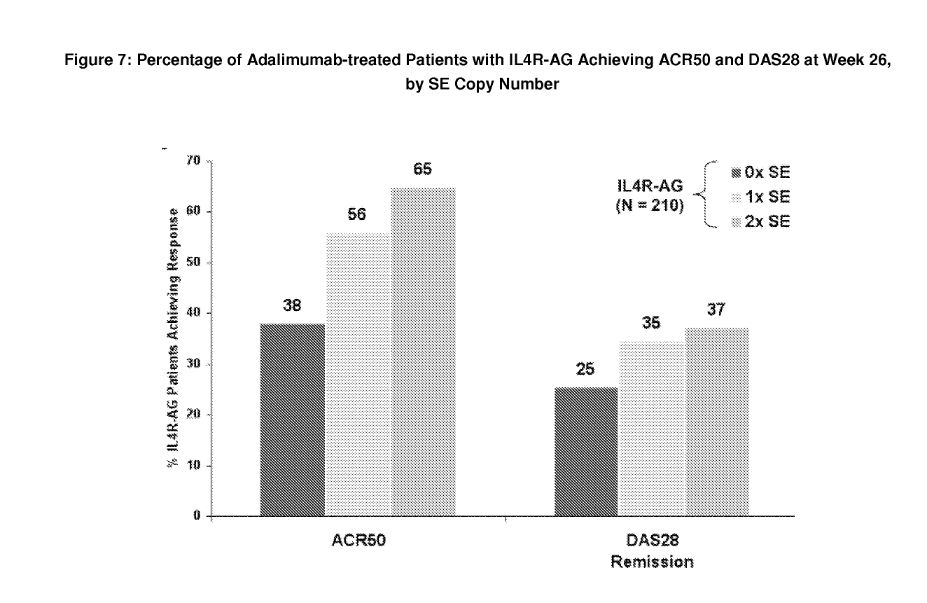

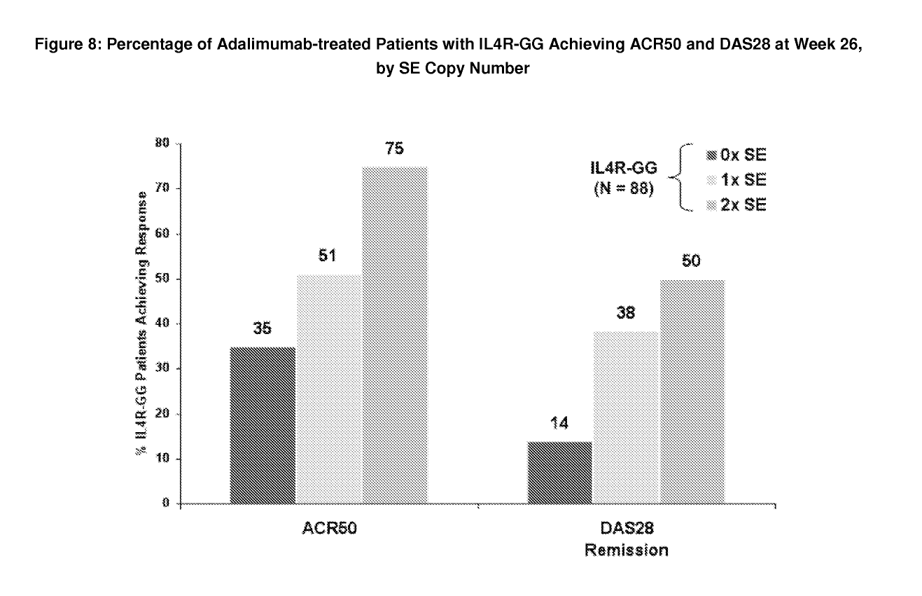

Consistent with findings from individual component analysis, ACR50 and DAS28 response rates in patients treated with ADA+MTX were enhanced in the presence of at least 1 copy of the SE, with the exception of those subjects who were homozygous for the IL-4R AA allele (FIGS. 6, 7, and 8). There was no consistent pattern of influence of SE and IL-4R allele combinations on response rates to PBO+MTX (data not shown).

In sum, the subjects in the treatment groups demonstrated a comparable distribution of 0, 1, or 2 copies of the HLA-DRB1 SE (PBO+MTX: 37%, 48%, 15%; ADA+MTX: 33%, 49%, 19%, respectively, P=0.28). Likewise, the IL-4R alleles, AA, AG, and GG, were distributed similarly between treatment groups (PBO+MTX: 29%, 52%, 20%; ADA+MTX: 33%, 47%, 20%, respectively, P=0.38). The FcγRIIb alleles, however, were dissimilarly appropriated between treatment groups, and no further analysis was conducted on this SNP (further analysis is presented in Example 4 below). The presence of the SE did not affect treatment response to MTX alone (eg, ACR50 of 40%, 33%, and 29% for 0, 1, or 2 copies, P=0.23). Conversely, treatment response rates were correspondingly enhanced with increasing copies of the SE in subjects receiving ADA+MTX (ACR50 of 42%, 53%, and 65% for 0, 1, 2 SE, P=0.004). Thus, the presence of 1 copy of the SE afforded a 20% increase in ACR50 for ADA+MTX subjects relative to the PBO+MTX group (P<0.001), and 2 copies of SE increased ACR50 in ADA+MTX by 36% over PBO+MTX (P<0.001). Similarly, clinical responses to MTX were not affected by IL-4R alleles, while treatment outcomes with ADA+MTX were enhanced in subjects with AA or AG IL-4R alleles. Examination of treatment responses for the SE and IL-4R allele combinations in the PBO+MTX group showed no alteration in responses by genotype, supporting the results obtained from analysis of the individual alleles. In the absence of the SE, IL-4R genotype affected treatment response to ADA+MTX, while the presence of the SE masked the effects of the IL-4R alleles (see Table 8 above).

### Conclusions

It was discovered that treatment with ADA+MTX offered a significant advantage for achieving ACR50 or DAS28 at 26 weeks of treatment compared with PBO+MTX. The HLA-DRB1 shared epitope demonstrated a significant effect on treatment response (ACR50) even when accounting for baseline demographic and disease state variables. In addition, IL-4R showed no appreciable effect on ACR50 or DAS28 Remission responses in multivariate regression. Thus, an understanding of the genetic components that contribute to treatment responses to TNF antagonists can assist in guiding therapeutic decisions.

In summary, clinical responses to adalimumab plus methotrexate were independently affected by both the HLA-DRB1 shared epitope and IL-4R alleles, while there was no impact of genotype on the response to methotrexate monotherapy. Thus, there was an interaction between the HLA-DRB1 shared epitope and IL-4R alleles in response to treatment with adalimumab plus methotrexate.

### Example 4

### Genetic Influence of HLA-DRB1, IL-4R, and FcγRIIb on Treatment Responses to Adalimumab Plus Methotrexate in Patients with Early Rheumatoid Arthritis: 26-Week Results of OPTIMA

Genetic factors are known to influence the manifestation, severity, and radiographic progression of rheumatoid arthritis. Their effect on responses to treatment with anti-TNF agents is unclear.

The objective of this study was to examine the response to adalimumab plus methotrexate (ADA+MTX) or placebo (PBO)+MTX following 26 weeks of treatment according to 3 candidate loci: the HLA-DRB1 shared epitope (SE), the IL-4R I50V variant, and the FcγRIIb I232T polymorphism.

### Methods

MTX-naïve pts ≧18 years old with RA<1 year and active disease (DAS28>3.2, ESR≧28 mm/h or CRP≧1.5 mg/dL), and either >1 erosions, RF+, or anti-CCP+ were randomized to ADA+MTX (N=515) or PBO+MTX (N=517) for 26 wks. This analysis presents clinical outcomes at 26 weeks by HLA-DRB1 SE copy number (0×, 1×, or 2×), IL-4R I50V (AA, AG, or GG), and FcγRIIb I232T (TT, TC, CC) alleles. Non-responder imputation was used to calculate the percent of patients achieving ACR20/50/70 and DAS28 low disease activity (LDA, DAS28<3.2) and remission (DAS28<2.6). Multiple logistic regression was used to assess the influence of potential confounding baseline variables. Categorical baseline explanatory variables included sex, smoker, RF (>50 or ≦50 IU), anti-CCP (≧3× or <3×ULN), CRP (≧1.5 or <1.5 mg/dl), and presence of erosions (0 or >0). Continuous values for baseline TJC68, SJC66, and DAS28 were also included.

### Results

In this substudy, genetic data were available for 451 and 443 patients randomized to PBO+MTX or ADA+MTX, respectively. The distribution of alleles was similar between treatment groups for SE and IL-4R. The FcγRIIb alleles were unequally distributed between the patients in both treatment arms (PBO-MTX) vs (ADA+MTX), as the PBO+MTX group had more TC and fewer CC FcγRIIb patients. Comparison between both treatment arms for FcγRIIb was not performed; however, it was possible to analyze the impact of all three FcγRIIb genotypes within one arm alone on response to their respective treatment.

For each locus, the baseline demographics were similar across alleles. A higher proportion of anti-CCP+ patients were noted among those with increasing copies of the SE.

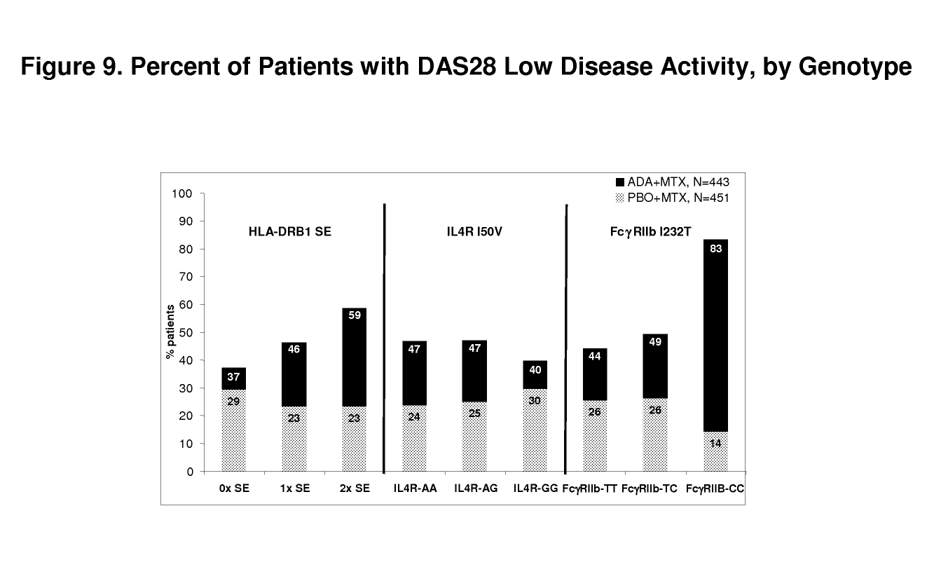

Responses to ADA+MTX increased with increasing SE copy number, decreased with IL-4R-GG, and increased with FcγRIIb-CC. An inverse pattern was detected for patients in the PBO+MTX group (e.g., DAS28<3.2, FIG. 9).

Because of this difference, multiple logistic regression was performed within each treatment group. In response to treatment with PBO+MTX, SE copy number demonstrated a significant negative effect on ACR20 and ACR50. IL-4R and FcγRIIb failed to show a significant association with 26-week responses to PBO+MTX.

In the model for ADA+MTX responses, SE copy number was significantly associated with achieving ACR20/50/70 and DAS28 LDA. Odds ratios showed that patients with 2 copies of the SE were approximately 2 times as likely to reach these targets as those with 0 SE alleles. IL-4R alone did not have an impact on 26-week treatment response to ADA+MTX. FcγRIIb-CC was significantly associated with achieving ACR70 and DAS28 remission, with odds ratios greater than 10 times that of FcγRIIb-TC. In combination, the effect of SE copy number was muted in the IL-4R-AA and FcγRIIb-TT wild type backgrounds, but apparent when at least 1 copy of either the IL-4R or FcγRIIb genetic variants were present.

In conclusion, regardless of genetic background, treatment response rates were higher for patients in the ADA+MTX group compared with PBO+MTX. Based on multiple logistic regression models, IL-4R alone was not associated with treatment outcomes. The HLA-DRB1 SE and FcγRIIb were independent significant positive predictors of response to ADA+MTX treatment. Potential interactions between these loci warrant further exploration of the role of these genetic components in response to anti-TNF agents, although it is clear from the data presented herein that HLA-DRB1 SE and FcγRIIb alone or in combination (and/or further in combination with IL-4R) are each predictors of a patient's response to treatment with a TNFa inhibitor.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

1. Disease Activity Score (DAS) in Rheumatoid Arthritis. [website] http://www.das-score.nl/www.das-score.nl/index.html. Accessed Aug. 21, 2006.
2. John M. Davis III y Eric L. Matteson. Reumatol Clin. 2009; 5(4): 143-146

1. A method of predicting the responsiveness of a subject having rheumatoid arthritis (RA) to treatment with a TNFα inhibitor, the method comprising determining the presence of an HLA-DRB1 shared epitope (HLA-DRB1 SE) allele in a sample from the subject, wherein the presence of at least one copy of the HLA-DRB1 SE allele indicates that the subject will be responsive to treatment with the TNFα inhibitor.

2. A method for treating a subject having rheumatoid arthritis (RA) comprising administering a TNFα inhibitor to the subject for the treatment of RA, provided that at least one copy of an HLA-DRB1 shared epitope (HLA-DRB1 SE) allele is present in a sample from the subject.

3. A method of determining whether a TNFα inhibitor will be effective for the treatment of a subject having rheumatoid arthritis (RA), the method comprising detecting the presence of at least one copy of an HLA-DRB1 shared epitope (HLA-DRB1 SE) allele in a sample from the subject, wherein the presence of the HLA-DRB1 SE allele indicates that the TNFα inhibitor will be effective for the treatment of RA in the subject.

4. (canceled)

5. (canceled)

6. The method of any one of claim 1, 2 or 3, further comprising determining the presence of an IL-4R I50 allele in a sample from the subject, wherein the presence of the IL-4R I50 allele (AA or AG) in the sample indicates that the subject will be responsive to treatment with the TNFα inhibitor.

7. The method of any one of claim 1, 2 or 3, further comprising determining the presence of two FcγRIIb T232 alleles (FcγRIIb-CC) in a sample from the subject, wherein the presence of two FcγRIIb T232 alleles (FcγRIIb-CC) in the sample indicates that the subject will be responsive to treatment with the TNFα inhibitor.

8. (canceled)

9. A method of predicting the responsiveness of a subject having RA to treatment with a TNFα inhibitor, the method comprising determining the copy number of an FcγRIIb T232 allele in a sample from the subject, wherein the presence of two copies of the FcγRIIb T232 allele (FcγRIIb-CC) indicates that the subject will be responsive to treatment with the TNFα inhibitor.

10. A method for treating a subject having rheumatoid arthritis (RA) comprising administering a TNFα inhibitor to the subject for the treatment of RA, provided that two copies of the FcγRIIb T232 allele (FcγRIIb-CC) are present in a sample from the subject.

11. A method of determining whether a TNFα inhibitor will be effective for the treatment of a subject having rheumatoid arthritis (RA), the method comprising determining the copy number of an FcγRIIb T232 allele in a sample from the subject, wherein the presence of two copies of the FcγRIIb T232 allele (FcγRIIb-CC) indicates that the TNFα inhibitor will be effective for the treatment of RA in the subject.

12. (canceled)

13. (canceled)

14. A method of predicting the responsiveness of a subject having RA to treatment with a TNFα inhibitor, the method comprising determining the number of copies of an IL-4R V50 allele in a sample from the subject, wherein the presence of two copies of the IL-4R V50 allele (GG) in the sample indicates that the subject will not be responsive to treatment with the TNFα inhibitor, unless the subject also has at least one copy of an HLA-DRB1 SE allele.

15. (canceled)

16. (canceled)

17. The method of any one of claims 1-3, 9-11, and 14, wherein the TNFα inhibitor is an anti-TNFα antibody, or antigen-binding portion thereof, or a fusion protein.

18. (canceled)

19. The method of claim 17, wherein the anti-TNFα antibody, or antigen-binding portion thereof, is selected from the group consisting of a human antibody, a chimeric antibody, a humanized antibody, and a multivalent antibody.

20. (canceled)

21. (canceled)

22. The method of claim 17, wherein the anti-TNFα antibody, or antigen-binding portion thereof, is an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

23. The method of claim 17, wherein the anti-TNFα antibody, or antigen-binding portion thereof, is an isolated human antibody with the following characteristics:
a) dissociates from human TNFα with a k_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

24. The method of claim 17, wherein the anti-TNFα antibody, or antigen-binding portion thereof, is an isolated human antibody with a light chain variable region (LCVR) comprising the amino acid sequence of SEQ ID NO: 1 and a heavy chain variable region (HCVR) comprising the amino acid sequence of SEQ ID NO: 2.

25. The method of any one of claims 1-3, 9-11, and 14, wherein the subject is diagnosed with RA with a disease duration of less than 1 year.

26. The method of any one of claims 1-3, 9-11, and 14, wherein the subject has a DAS28 of >3.2.

27. The method of any one of claims 1-3, 9-11, and 14, wherein the subject is further administered MTX.

28. The method of any one of claims 1, 3, 9 and 11, wherein the method determines or predicts clinical responsiveness in the subject.

29. A kit for predicting a subject's responsiveness to a TNFα inhibitor for the treatment of rheumatoid arthritis (RA), the kit comprising
a means for determining the presence of an HLA-DRB1 SE allele in a sample from the subject, and
instructions for recommended treatment for the subject based on the presence of the HLA-DRB1 SE allele, wherein the presence of the HLA-DRB1 SE allele indicates that the subject will be responsive to treatment of RA with the TNFα inhibitor.

30. The kit of claim 29, wherein the means for determining the presence of the HLA-DRB1 SE allele comprises either
a nucleic acid that hybridizes to a nucleic acid molecule encoding HLA-DRB1 SE, or a portion thereof containing the SE region, or
an antibody which specifically binds to a protein corresponding to HLA-DRB1 SE.

31. The kit of claim 29 or 30, further comprising
a means for detecting the presence of an IL-4R I50 allele in the sample from the subject, and
instructions for recommended treatment for the subject based on the presence of the IL-4R I50 allele, wherein the combined presence of the IL-4R I50 allele and the HLA-DRB1 SE allele indicates that the subject will be responsive to treatment of RA with the TNFα inhibitor.

32. A kit for predicting or assessing a subject's responsiveness to a TNFα inhibitor for the treatment of rheumatoid arthritis (RA), the kit comprising
a) a means for determining the presence of an FcγRIIb T232 allele in a sample from the subject, and
b) instructions for recommended treatment for the subject based on the presence of two FcγRIIb T232 alleles (FcγRIIb-CC), wherein the presence of two FcγRIIb T232 alleles indicates the subject will be responsive to treatment of RA with the TNFα inhibitor.

33. The kit of claim 32, wherein the means for determining the presence of the FcγRIIb T232 allele comprises either
a nucleic acid that hybridizes to a nucleic acid molecule encoding FcγRIIb T232, or a portion thereof containing the I232T SNP, or
an antibody which specifically binds to a protein corresponding to an FcγRIIb T232 protein.

34. The kit of claim 32 or 33, further comprising
a means for detecting the presence of an IL-4R I50 allele in the sample from the subject, and
instructions for recommended treatment for the subject based on the presence of the IL-4R I50 allele, wherein the combined presence of the IL-4R I50 allele and the FcγRIIb-CC allele indicates that the subject will be responsive to treatment or RA with the TNFα inhibitor.

35. The kit of claim 34, further comprising
a means for detecting the presence of an HLA-DRB1 SE allele in the sample from the subject, and
instructions for recommended treatment for the subject based on the presence of the HLA-DRB1 SE allele, wherein the combined presence of the FcγRIIb-CC allele, the IL-4R I50 allele, and the HLA-DRB1 SE allele indicates that the subject will be responsive to treatment of RA with the TNFα inhibitor.

36. The kit of claim 32 or 33, further comprising
a means for detecting the presence of an HLA-DRB1 SE allele in the sample from the subject, and
instructions for recommended treatment for the subject based on the presence of the HLA-DRB1 SE allele, wherein the combined presence of the FcγRIIb-CC allele and the HLA-DRB1 SE allele indicates that the subject will Be responsive to treatment of RA with the TNFα inhibitor.

37. (canceled)

38. The kit of claim 29 or 32, wherein the TNFα inhibitor is an anti-TNFα antibody, or antigen-binding portion thereof, or a fusion protein.

39. (canceled)

40. The kit of claim 38, wherein the anti-TNFα antibody, or antigen-binding portion thereof, is selected from the group consisting of a human antibody, a chimeric antibody, a humanized antibody, and a multivalent antibody.

41. (canceled)

42. (canceled)

43. The kit of claim 38, wherein the anti-TNFα antibody, or antigen-binding portion thereof, is an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

44. The kit of claim 38, wherein the anti-TNFα antibody, or antigen-binding portion thereof, is an isolated human antibody with the following characteristics:
a) dissociates from human TNFα with a k_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

45. The kit of claim 38, wherein the anti-TNFα antibody, or antigen-binding portion thereof, is an isolated human antibody with a light chain variable region (LCVR) comprising the amino acid sequence of SEQ ID NO: 1 and a heavy chain variable region (HCVR) comprising the amino acid sequence of SEQ ID NO: 2.
